(19) United States
(12) Patent Application Publication
(10) Pub. No.: US 2024/0105342 A1
(43) Pub. Date: Mar. 28, 2024

(54) DYNAMIC DATA RESAMPLING BASED ON MEDICAL RELEVANCE

(71) Applicant: Eye To Eye Telehealth, Inc., Cheyenne, WY (US)

(72) Inventors: Preston Eugene Smits, Denver, CO (US); David James Smits, Cheyenne, WY (US)

(73) Assignee: Eye To Eye Telehealth, Inc, Cheyenne, WY (US)

(21) Appl. No.: 18/373,105

(22) Filed: Sep. 26, 2023

Related U.S. Application Data
(60) Provisional application No. 63/377,194, filed on Sep. 26, 2022.

(51) Int. Cl. G16H 50/30; G16H 50/20

(52) U.S. Cl. CPC G16H 50/30; G16H 50/20

(57) ABSTRACT

A computer system that provides feedback regarding a set of data is described. Notably, the computer system may determine whether the set of data is medically meaningful. For example, the set of data may include intraocular pressure measurements during a time interval. When the measurements are at incorrect times of day (such as relative to a treatment time), the set of data may not be medically meaningful. When the set of data is not medically meaningful, the computer system may provide a request for additional data, such as a different type of data, a different sampling time for the additional data, etc. Otherwise, when the set of data is medically meaningful, the computer system may analyze the set of data to determine analysis results, and then may selectively provide feedback associated with glaucoma of the patient or treatment of the patient based at least in part on the analysis results.

### CROSS REFERENCE TO RELATED APPLICATIONS

This application claims priority under 35 U.S.C. 119(e) to U.S. Provisional Application Ser. No. 63/377,194, “Dynamic Data Resampling Based on Medical Relevance,” filed on Sep. 26, 2022, by Preston Eugene Smits, et al. the contents of which are herein incorporated by reference.

### FIELD

The described embodiments relate to providing feedback regarding a set of data. Notably, the described embodiments relate to selectively providing a request for additional data when the set of data is not medically meaningful.

### BACKGROUND

The increased availability of consumer monitoring devices is allowing patients to collect data associated with their medical conditions. For example, cellular telephones, smart watches and fitness trackers allow patients to collect information about their symptoms and treatments, such as whether or not a patient used a medication, when the medication was used, etc.

In principle, these patient-reported and patient-acquired datasets (which are henceforth referred to as ‘patient-acquired datasets’) can allow healthcare providers to better understand patients' medical conditions, patient behaviors and lifestyles, patient compliance with heathcare-provider recommendations and/or the efficacy of current treatments. Notably, by providing data between patient appointments with their healthcare providers (which are typically infrequent), the patient-acquired datasets can significantly improve the situation awareness of the healthcare providers regarding the circumstances of specific patients, thereby allowing the healthcare providers to better manage care of these patients.

However, in practice, it is often difficult to obtain these benefits. Notably, patient-acquired data is often incomplete or inadequate. These deficiencies may undermine the value of the patient-acquired datasets for healthcare providers, which makes it more difficult for healthcare providers to manage patient care. Consequently, healthcare providers still usually make recommendations (such as treatment changes) based on limited or incomplete data, which necessitates a trial-and-error approach to patient care, and increases patient suffering, patient mortality and associated costs.

### SUMMARY

A computer system that selectively provides feedback is described. This computer system includes: an interface circuit; a computation device coupled to the interface circuit; and memory, coupled to the computation device, storing program instructions. During operation, the computer system obtains a set of data associated with an individual, where the set of data includes or corresponds to a history of glaucoma diagnostic or glaucoma treatment data for the individual during a time interval. Then, the computer system determines whether the set of data is medically meaningful for the individual based at least in part on one or more of: a number of data instances in the set of data, a number of types of data in the set of data, or sampling times of the data instances. When the set of data is determined to not be medically meaningful for the individual, the computer system provides, addressed to an electronic device, a request for additional data, where the additional data includes one or more of: an additional data instance, an additional type of data that is different from the types of data, or additional glaucoma diagnostic or glaucoma treatment data at one or more different sampling times than the sampling times of the data instances. Alternatively, when the set data is determined to be medically meaningful for the individual, the computer system: analyzes the set of data to compute analysis results; and selectively provides, addressed to the electronic device and/or a second electronic device, the feedback associated with glaucoma of the individual or glaucoma treatment of the individual, where the feedback is selectively provided based at least in part on analysis results.

Note that the obtaining may include: performing measurements of the glaucoma diagnostic or the glaucoma treatment data; accessing the glaucoma diagnostic or the glaucoma treatment data stored in the memory; and/or receiving, associated with the electronic device, the glaucoma diagnostic or the glaucoma treatment data.

Moreover, the electronic device may be associated with the individual and the second electronic device may be associated with a clinician (which is sometimes referred to as a healthcare provider).

Furthermore, the determining and/or the analyzing may be performed using a pretrained model. For example, the pretrained model may include a supervised-learning model or a neural network.

Additionally, the feedback may include a treatment recommendation, such as a change to a current treatment of the individual.

In some embodiments, the determining and/or the analysis may be based at least in part on one or more of: a glaucoma stage or risk of the individual; a prescribed medication used by the individual; one or more surgical, medical, or laser treatments or interventions performed on an individual; a clinician-defined alert criterion; a clinical insight or question; and/or demographic information of the individual.

Note that the types of data may include: intraocular pressure, a visual field test, Optical Coherence Tomography (OCT), pachymetry, corneal hysteresis, medical treatment or surgical or laser intervention performed by a clinician, a patient-supplied input (such as a medication side effect), medication compliance of the individual, and/or times when a prescribed medication is used by the individual.

Another embodiment provides a monitoring device that performs the measurements of the glaucoma diagnostic or the glaucoma treatment data

Another embodiment provides the electronic device.

Another embodiment provides the second electronic device.

Another embodiment provides a computer-readable storage medium for use with the computer system, the electronic device or the second electronic device. When executed by the computer system, the electronic device or the second electronic device, this computer-readable storage medium causes the computer system, the electronic device or the second electronic device to perform at least some of the aforementioned operations or counterparts to at least some of the aforementioned operations.

Another embodiment provides a method, which may be performed by the computer system, the electronic device or the second electronic device. This method includes at least some of the aforementioned operations or counterparts to at least some of the aforementioned operations.

This Summary is provided for purposes of illustrating some exemplary embodiments, so as to provide a basic understanding of some aspects of the subject matter described herein. Accordingly, it will be appreciated that the above-described features are examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE FIGURES

FIG. 1 is a block diagram illustrating an example of communication among a monitoring device, a computer system and one or more electronic devices in accordance with an embodiment of the present disclosure.

FIG. 2 is a flow diagram illustrating an example of a method for selectively providing feedback using the computer system in FIG. 1 in accordance with an embodiment of the present disclosure.

FIG. 3 is a drawing illustrating an example of communication among a monitoring device, a computer system and one or more electronic devices in FIG. 1 in accordance with an embodiment of the present disclosure.

FIG. 4 is a drawing illustrating an example of intraocular pressure (IOP) measurements and threshold values in accordance with an embodiment of the present disclosure.

FIG. 5 is a drawing illustrating an example of IOP measurements and threshold values in accordance with an embodiment of the present disclosure.

FIG. 6 is a drawing illustrating an example of IOP measurements as a function of time and threshold values in accordance with an embodiment of the present disclosure.

FIG. 7 is a drawing illustrating an example of IOP measurements and threshold values in accordance with an embodiment of the present disclosure.

FIG. 8 is a drawing illustrating an example of IOP measurements as a function of time and threshold values in accordance with an embodiment of the present disclosure.

FIG. 9 is a drawing illustrating an example of visual field index measurements, IOP measurements and medication compliance as a function of time in accordance with an embodiment of the present disclosure.

FIG. 10 is a drawing illustrating an example of IOP measurements as a function of time and threshold values in accordance with an embodiment of the present disclosure.

FIG. 11 is a block diagram illustrating an example of an electronic device in accordance with an embodiment of the present disclosure.

Note that like reference numerals refer to corresponding parts throughout the drawings. Moreover, multiple instances of the same part are designated by a common prefix separated from an instance number by a dash.

### DETAILED DESCRIPTION

A computer system that provides feedback regarding a set of data for glaucoma is described. Notably, the computer system may determine whether the set of data is medically meaningful. For example, the set of data may include intraocular pressure measurements during a time interval. When the measurements are at incorrect times of day (such as relative to when a patient uses a glaucoma treatment, such as eye drops), the set of data may not be medically meaningful. When the set of data is not medically meaningful, the computer system may provide a request for additional data, such as a different type of data, a different sampling time for the additional data, etc. Otherwise, when the set of data is medically meaningful, the computer system may analyze the set of data to determine analysis results, and then may selectively provide feedback associated with glaucoma of the patient or treatment of the patient based at least in part on the analysis results.

By selectively requesting additional data, these monitoring techniques may improve the medical relevance of the set of data and, thus, may improve the accuracy of the feedback. For example, if needed, the additional data may ensure there is sufficient time, diversity of data and/or appropriate sampling (such as relative to when the patent uses the glaucoma treatment). The improved data quality and/or the increased sampling frequency may facilitate improved (e.g., accurate and appropriate) subsequent analysis and/or feedback, such as a recommendation to change or modify the glaucoma treatment, or more accurate diagnostic information about one or more potential medical conditions. Consequently, the monitoring techniques may help improve patient care and patient well-being and outcomes, which may reduce patient suffering, mortality and associated costs (such as direct costs of treatment and indirect costs of lost productivity, etc.).

Moreover, the ability to selectively acquire more medically meaningful data may improve the operation or function of the computer system. Notably, the improved data quality and/or the increased sampling frequency may allow the computer system to analyze the data and/or to provide the feedback while using fewer resources (such as less processor, memory and/or network resources). For example, the improved data quality and/or the increased sampling frequency may allow the computer system to analyze the data more rapidly or successfully (stated differently, the data that is not medically meaningful may not be able to be analyzed or may provide erroneous results). Furthermore, improved data quality and/or the increased sampling frequency may allow the computer system to analyze smaller datasets, which may also consume fewer resources.

In the discussion that follows, electronic devices, computers and/or servers (which may be local or remotely located from each other) may communicate packets or frames in accordance with a wired communication protocol and/or a wireless communication protocol. The wireless communication protocol may include: a wireless communication protocol that is compatible with an Institute of Electrical and Electronics Engineers (IEEE) 802.11 standard (which is sometimes referred to as ‘Wi-Fi®,’ from the Wi-Fi Alliance of Austin, Texas), Bluetooth, Bluetooth low energy, a cellular-telephone network or data network communication protocol (such as a third generation or 3G communication protocol, a fourth generation or 4G communication protocol, e.g., Long Term Evolution or LTE (from the 3rd Generation Partnership Project of Sophia Antipolis, Valbonne, France), LTE Advanced or LTE-A, a fifth generation or 5G communication protocol, a low-power wide area network (LPWAN) cellular technology (such as CAT-M1, narrow band Internet of things, etc.), or other present or future developed advanced cellular communication protocol), and/or another type of wireless interface (such as another wireless-local-area-network interface). For example, an IEEE 802.11 standard may include one or more of: IEEE 802.11a, IEEE 802.11b, IEEE 802.11g, IEEE 802.11-2007, IEEE 802.11n, IEEE 802.11-2012, IEEE 802.11-2016, IEEE 802.11ac, IEEE 802.11ax, IEEE 802.11ba, IEEE 802.11be, or other present or future developed IEEE 802.11 technologies. Moreover, the wired communication protocol may include a wired communication protocol that is compatible with an IEEE 802.3 standard (which is sometimes referred to as ‘Ethernet’), e.g., an Ethernet II standard. However, a wide variety of communication protocols may be used. In the discussion that follows, Bluetooth and Ethernet are used as illustrative examples.

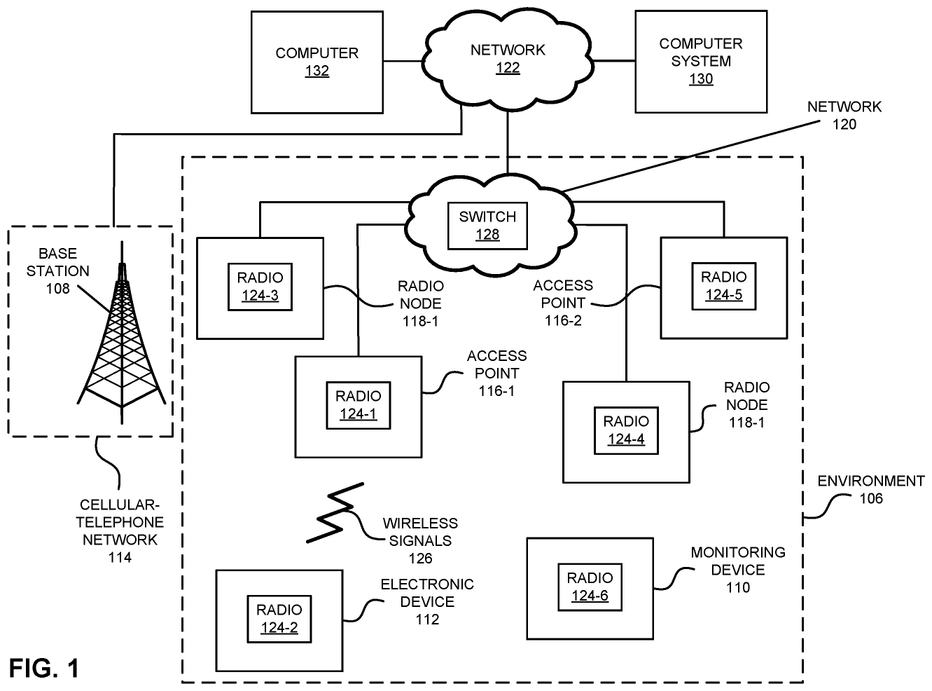

We now describe some embodiments of the monitoring techniques. FIG. 1 presents a block diagram illustrating an example of communication between a monitoring device 110 and an electronic device 112 (such as a cellular telephone, a portable electronic device, or another type of electronic device, etc.). Moreover, electronic device 112 may optionally communicate via a cellular-telephone network 114 (which may include a base station 108), one or more access points 116 (which may communicate using Wi-Fi) in a wireless local area network (WLAN) and/or radio node 118 (which may communicate using LTE or a cellular-telephone data communication protocol) in a small-scale network (such as a small cell). For example, radio node 118 may include: an Evolved Node B (eNodeB), a Universal Mobile Telecommunications System (UMTS) NodeB and radio network controller (RNC), a New Radio (NR) gNB or gNodeB (which communicates with a network with a cellular-telephone communication protocol that is other than LTE), etc. In the discussion that follows, an access point, a radio node or a base station are sometimes referred to generically as a ‘communication device.’ Moreover, one or more base stations (such as base station 108), access points 116, and/or radio node 118 may be included in one or more networks, such as: a WLAN, a small cell, a local area network (LAN) and/or a cellular-telephone network. In some embodiments, access points 116 may include a physical access point and/or a virtual access point that is implemented in software in an environment of an electronic device or a computer.

Furthermore, electronic device 112 may optionally communicate with computer system 130 (which may include one or more computers or servers, and which may be implemented locally or remotely to provide storage and/or analysis services) using a wired communication protocol (such as Ethernet) via network 120 and/or 122. Note that networks 120 and 122 may be the same or different networks. For example, networks 120 and/or 122 may be a LAN, an intra-net or the Internet. In some embodiments, the wired communication protocol may include a secured connection over transmission control protocol/Internet protocol (TCP/IP) using hypertext transfer protocol secure (HTTPS). Additionally, in some embodiments, network 120 may include one or more routers and/or switches (such as switch 128).

Electronic device 112 and/or computer system 130 may implement at least some of the operations in the monitoring techniques. Notably, as described further below, electronic device 112 and/or computer system 130 may perform at least some of the analysis of measurement data acquired by monitoring device 110, and may provide feedback information to monitoring device 110, electronic device 112 and/or computer 132.

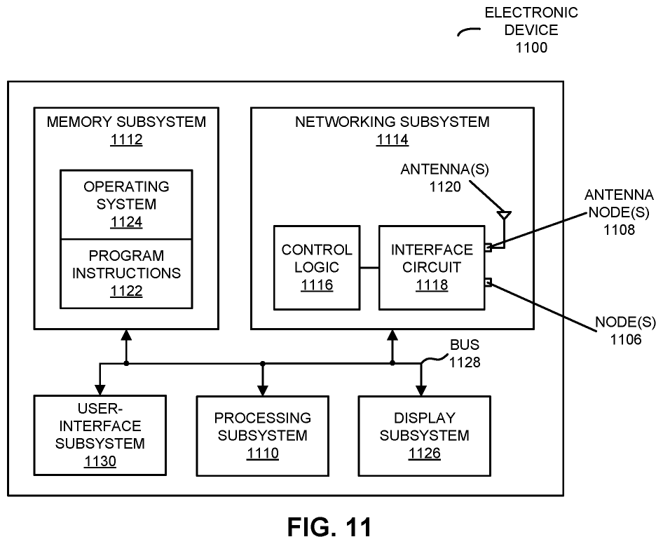

As described further below with reference to FIG. 11, base station 108, monitoring device 110, electronic device 112, access points 116, radio node 118, switch 128, computer system 130 and/or computer system 132 may include subsystems, such as a networking subsystem, a memory subsystem and a processor subsystem. In addition, monitoring device 110, electronic device 112, access points 116 and radio node 118 may include radios 124 in the networking subsystems. More generally, monitoring device 110, electronic device 112, access points 116 and radio node 118 can include (or can be included within) any electronic devices with the networking subsystems that enable monitoring device 110, electronic device 112, access points 116 and radio node 118 to wirelessly communicate with one or more other electronic devices. This wireless communication can comprise transmitting access on wireless channels to enable electronic devices to make initial contact with or detect each other, followed by exchanging subsequent data/management frames (such as connection requests and responses) to establish a connection, configure security options, transmit and receive frames or packets via the connection, etc.

During the communication in FIG. 1, base station 108, monitoring device 110, electronic device 112, access points 116, radio node 118, computer system 130 and/or computer system 132 may wired or wirelessly communicate while: transmitting access requests and receiving access responses on wired or wireless channels, detecting one another by scanning wireless channels, establishing connections (for example, by transmitting connection requests and receiving connection responses), and/or transmitting and receiving frames or packets (which may include information as payloads).

As can be seen in FIG. 1, wireless signals 126 (represented by a jagged line) may be transmitted by radios 124 in, e.g., access points 116 and/or radio node 118 and monitoring device 110 or electronic device 112. For example, radio 124-1 in access point 116-1 may transmit information (such as one or more packets or frames) using wireless signals 126. These wireless signals are received by radio 124-2 in electronic device 112. This may allow access point 116-1 to communicate information to other access points 116 and/or electronic device 112. Note that wireless signals 126 may convey one or more packets or frames. While FIG. 1 illustrates monitoring device 110 with an electronic device that is capable of wireless communication, in other embodiments monitoring device 110 may or may not be an electronic device, and thus may or may not be capable of wireless communication.

In the described embodiments, processing a packet or a frame in one or more electronic devices in monitoring device 110, electronic device 112, access points 116, radio node 118 and/or computer system 130 may include: receiving the wireless or electrical signals with the packet or the frame; decoding/extracting the packet or the frame from the received wireless or electrical signals to acquire the packet or the frame;

and processing the packet or the frame to determine information contained in the payload of the packet or the frame.

Note that the wired and/or wireless communication in FIG. 1 may be characterized by a variety of performance metrics, such as: a data rate for successful communication (which is sometimes referred to as ‘throughput’), an error rate (such as a retry or resend rate), a mean-squared error of equalized signals relative to an equalization target, intersymbol interference, multipath interference, a signal-to-noise ratio, a width of an eye pattern, a ratio of number of bytes successfully communicated during a time interval (such as 1-10 s) to an estimated maximum number of bytes that can be communicated in the time interval (the latter of which is sometimes referred to as the ‘capacity’ of a communication channel or link), and/or a ratio of an actual data rate to an estimated data rate (which is sometimes referred to as ‘utilization’). While instances of radios 124 are shown in components in FIG. 1, one or more of these instances may be different from the other instances of radios 124.

In some embodiments, wireless communication between components in FIG. 1 uses one or more bands of frequencies, such as: 900 MHz, 2.4 GHz, 5 GHz, 6 GHz, 60 GHz, the Citizens Broadband Radio Spectrum or CBRS (e.g., a frequency band near 3.5 GHz), and/or a band of frequencies used by LTE or another cellular-telephone communication protocol or a data communication protocol. Note that the communication between electronic devices may use multi-user transmission (such as orthogonal frequency division multiple access or OFDMA).

Although we describe the network environment shown in FIG. 1 as an example, in alternative embodiments, different numbers or types of electronic devices and/or monitoring devices may be present. For example, some embodiments comprise more or fewer electronic devices or monitoring devices. In some embodiments, a monitoring device may be integrated into or included in another electronic device. As another example, in another embodiment, different electronic devices are transmitting and/or receiving packets or frames.

While FIG. 1 illustrates computer system 130 at a particular location, in other embodiments at least a portion of computer system 130 is implemented at more than one location. Thus, in some embodiments, computer system 130 is implemented in a centralized manner, while in other embodiments at least a portion of computer system 130 is implemented in a distributed manner.

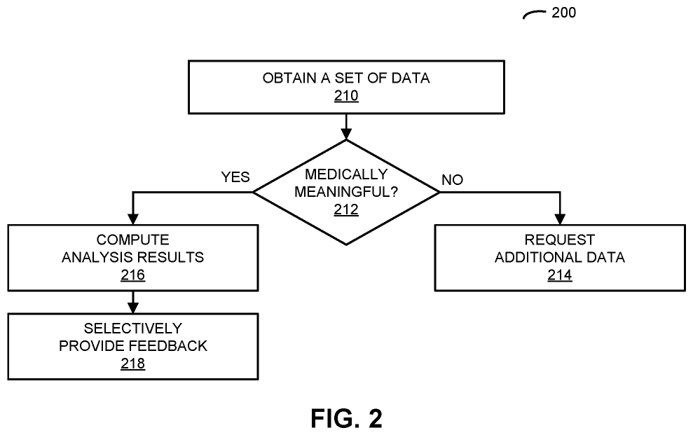

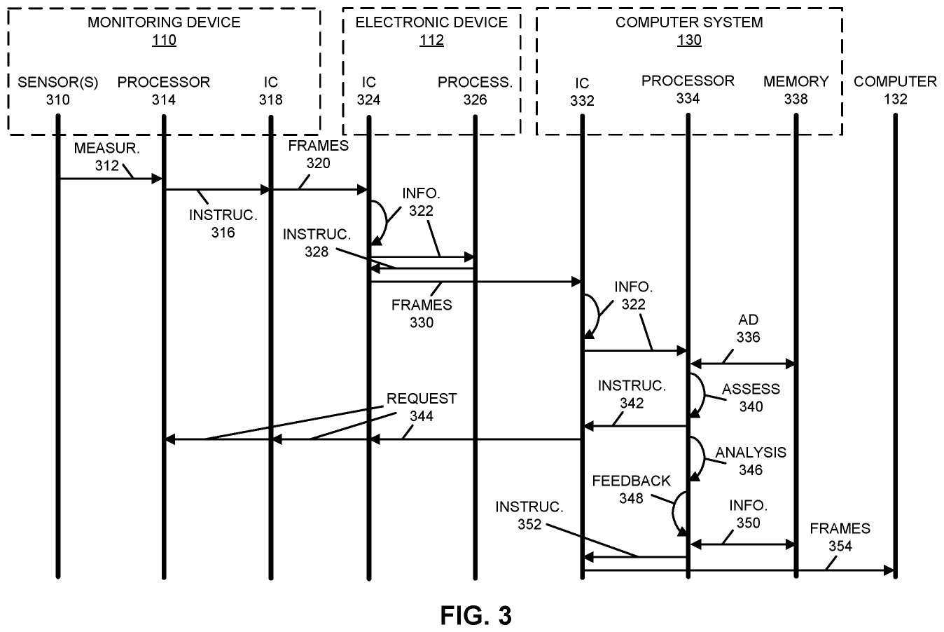

As discussed previously, patient-acquired data is often incomplete or inadequate. Moreover, as described further below with reference to FIGS. 2-3, in order to address these challenges, monitoring device 110, electronic device 112 and/or computer system 130 may perform the monitoring techniques. Notably, monitoring device 110 (which may be associated with an individual) may perform measurements on the individual, such as intraocular pressure measurements and, more generally, glaucoma diagnostic or glaucoma treatment data. Alternatively, monitoring device 110 could be in-office diagnostic equipment that the patient does not own or operate, rather it is operated and measurements are taken by the clinician or office staff, and these measurements may be accessed by computer system 130 in the monitoring techniques. For example, the measurements may include a variety of types of data, such as: a visual field test, OCT, pachymetry, corneal hysteresis, medication compliance of the individual (which may be measured using an accelerometer and/or a sensor on an eyedrop vial or cartridge), and/or times when a prescribed medication is used by the individual, or a surgical, implanted medication, or laser treatment is performed. In some embodiments, IOP may be measured using rebound tonometry, such as using a metal wire with a plastic tip that is pressed into or collides with the surface of the eye using a solenoid to measure the contact time, which corresponds to the IOP. In other embodiments, IOP may be measured using a different type of tonometer, such as a non-contact device or implantable IOP sensor. Note that the measured or patient-supplied input may include patient inputs to electronic device 112 that a medication is hurting or irritating them, (such as an adverse drug effect or another medical problem).

Then, the measurements may be assessed and/or analyzed to determine additional information. The assessment and/or analysis may, at least in part, be performed locally (e.g., by monitoring device 110), remotely (e.g., by electronic device 112 and/or computer system 130), or jointly by monitoring device 110, electronic device 112 and/or computer system 130. For example, monitoring device 110 may provide information specifying the measurements via Bluetooth or Bluetooth low energy to electronic device 112 (which may be associated with the individual). Then, electronic device 112 may perform at least a portion of the assessment and/or the analysis. Alternatively or additionally, after receiving the information specifying the measurements, electronic device 112 may provide, via networks 120 and 122, the information to computer system 130, which may perform at least a portion of the assessment and/or analysis. As noted previously, the communication among monitoring device 110, electronic device 112 and/or computer system 130 may be secure (e.eg., encrypted and/or via a tunnel) in order to protect personal and/or medical information.

For example, after receiving the information specifying the measurements, computer system 130 may optionally aggregate the measurements with one or more previous instances of measurements to create a set of data associated with an individual, such as a history of glaucoma diagnostic or glaucoma treatment data for the individual during a time interval (such as a week, a month, a time interval since a previous appointment with a healthcare provider of the individual, etc.). Then, computer system 130 may determine whether the set of data is medically meaningful for the individual based at least in part on one or more of: a number of data instances in the set of data, a number of types of data in the set of data, or sampling times of the data instances. When the set of data is determined to not be medically meaningful for the individual, computer system 130 may provide, addressed to monitoring device 110, another or a different type of monitoring device (not shown) and/or electronic device 112, a request for additional data, where the additional data may include one or more of: an additional data instance, an additional type of data that is different from the types of data, or additional glaucoma diagnostic or glaucoma treatment data at one or more different sampling times than the sampling times of the data instances. In response to the request, monitoring device 110, the other or the different monitoring device, electronic device 112 and/or computer system 130 may repeat some or all of the preceding operations, e.g., until a medically meaningful set of data is obtained. In some embodiments, in response to the request, electronic device 112 may read additional measurements stored in memory in monitoring device 110 or the other or the different monitoring device and/or the individual (or patient) may upload the additional measurements stored in the memory in monitoring device 110 or the other or the different monitoring device (such as when monitoring device 110 includes an implantable IOP sensor).

Alternatively, when the set data is determined to be medically meaningful for the individual, computer system 130 may: analyzes the set of data to compute analysis results; and selectively provide, addressed to monitoring device 110, electronic device 112 and/or computer 132 (such as a computer associated with a clinician or a healthcare provider of the individual), feedback associated with glaucoma of the individual or glaucoma treatment of the individual, where the feedback is selectively provided based at least in part on analysis results. Note that the feedback may include a treatment recommendation, such as a change to a current treatment of the individual. In some embodiments, the assessment and/or the analysis may be based at least in part on one or more of: a glaucoma stage or risk of the individual; a prescribed medication used by the individual; one or more surgical, medical, or laser treatments or interventions performed on an individual; a clinician-defined alert criterion; a relevant clinical insight or question; and/or demographic information of the individual.

After receiving analysis results and/or the feedback, monitoring device 110, electronic device 112 and/or computer 132 may provide or present information corresponding to the analysis results and/or the feedback. For example, electronic device 112 and/or computer 132 may display a user interface information (such as one or more graphs) corresponding to the measurements, the analysis results, the feedback, and/or additional information (such as diagnostic information, etc.). A healthcare provider may use the analysis results and/or the feedback to add to, change or modify a treatment of the individual for a medical condition (such as glaucoma). Notably, the healthcare provider (such as a physician or a nurse practitioner) may prescribe or recommend additional treatment(s) or intervention(s) and/or may diagnose one or more medical conditions.

Alternatively or additionally, monitoring device 110 may provide information corresponding to the feedback, such as signals (e.g., selectively illuminating one or more lights or outputting sound or a tone) or instructions (e.g., verbal instructions) to guide the individual (such as when the individual should measure glaucoma diagnostic or glaucoma treatment data). For example, the feedback may include selective illumination of a green, yellow or red light emitting diode (LED) to indicate when to perform one or more measurements.

In some embodiments, monitoring device 110, electronic device 112 and/or computer system 130 may compute diagnostic information associated with a medical condition (such as glaucoma) based at least in part on the set of data. This diagnostic information may be provided or presented by monitoring device 110, electronic device 112 and/or computer 132. For example, electronic device 112 and/or computer 132 may display information corresponding to the diagnostic information in the user interface.

Moreover, in some embodiments, analysis results, diagnostic information and/or feedback may be aggregated over time, e.g., by computer system 130, into a history over a time interval or a summary report. This aggregated information may be reported to a user of monitoring device 110 and/or electronic device 112 (e.g., the individual). Alternatively or additionally, the history or summary report may, with user approval/authorization, be provided to a healthcare provider, such as to computer 132. Note that the history or summary report may be used in a clinical trial or in analysis of a population or group of individuals.

The assessment and/or analysis of the set of data to calculate the analysis results, compute of the diagnostic information, and/or determine or select the feedback (e.g., from a set of predefined or predetermined types of feedback) may be performed in a variety of ways. For example, one or more of the aforementioned operations may involve statistical calculations and/or comparisons with baseline information for the individual and/or a population or group of individuals (such as historical values stored by computer system 130).

Alternatively or additionally, the assessment and/or analysis of the set of data to calculate the analysis results, compute of the diagnostic information, and/or determine or select the feedback may be performed using an analysis model that is pretrained or predetermined using a machine-learning technique (such as a supervised learning technique, an unsupervised learning technique, e.g., a clustering technique, and/or a neural network) and a training dataset. For example, the analysis model may include a classifier or a regression model that was trained using: a support vector machine technique, a classification and regression tree technique, logistic regression, LASSO, linear regression, a neural network technique (such as a convolutional neural network technique, an autoencoder neural network or another type of neural network technique) and/or another linear or nonlinear supervised-learning technique. The analysis model may use requests for additional data, one or more instances of measurements (such as the glaucoma diagnostic or glaucoma treatment data), analysis results and/or feedback as inputs and may output: the analysis results, the diagnostic information, and/or the feedback (such as a recommendation for a change or a modification to a treatment). Note that computer system 130 may dynamically retrain the analysis model based at least in part on updates to the training dataset (such as recent requests for additional data, one or more instances of measurements, analysis results and/or feedback), and then may optionally provide an updated analysis model to: monitoring device 110 and/or electronic device 112.

In these ways, the monitoring techniques may facilitate improved real-world monitoring of a medical condition (such as glaucoma), and may provide real-time and real-world feedback on how to improve patient care. Furthermore, the monitoring techniques may provide diagnostic information and additional information that allows: medical conditions to be diagnosed, current therapies and interventions to be assessed, and/or additional therapies and interventions to be identified. These capabilities may assist patients and healthcare providers (such as medical professionals), and may improve the health and well-being of patients.

While the preceding discussion illustrated the monitoring techniques with monitoring device 110 acquiring the measurements that are assessed and/or analyzed, in some embodiments computer system 130 may perform a retrospective assessment and/or analysis of glaucoma diagnostic or the glaucoma treatment data stored in memory. Moreover, while the preceding embodiments illustrated the use of the monitoring techniques in conjunction with glaucoma and associated measurements (such as the glaucoma diagnostic or glaucoma treatment data), in other embodiments the monitoring techniques may be used with a wide variety of medical conditions and types of measurements. Furthermore, while the preceding embodiments illustrated the use of the monitoring techniques based on measurements and/or patient-reported data, in other embodiments the monitoring techniques may also leverage or use additional types of data, such as electronic medical records with information about the medical history of the individual and/or one or more other individuals (such as one or more individuals in a population that includes the individual).

We now describe embodiments of the method. FIG. 2 presents a flow diagram illustrating an example of a method 200 for selectively providing feedback, which may be performed by a computer system (such as computer system 130 in FIG. 1) and, more generally, an electronic device (such as monitoring device 110 or electronic device 112 in FIG. 1). During operation, the computer system may obtain a set of data (operation 210) associated with an individual, where the set of data includes or corresponds to a history of glaucoma diagnostic or glaucoma treatment data for the individual during a time interval. Note that the obtaining (operation 210) may include: performing measurements of the glaucoma diagnostic or the glaucoma treatment data; accessing the glaucoma diagnostic or the glaucoma treatment data stored in the memory; and/or receiving, associated with the electronic device, the glaucoma diagnostic or the glaucoma treatment data.

Then, the computer system may determine whether the set of data is medically meaningful (operation 212) for the individual based at least in part on one or more of: a number of data instances in the set of data, a number of types of data in the set of data, or sampling times of the data instances. Note that the types of data may include: intraocular pressure, a visual field test, OCT, pachymetry, corneal hysteresis, medical treatment or surgical or laser intervention performed by a clinician, a patient-supplied input (such a medication side effect or an adverse drug effect, and, more generally, information that indicates that a medication is hurting or irritating a patient), medication compliance of the individual, and/or times when a prescribed medication is used by the individual.

When the set of data is determined to not be medically meaningful (operation 212) for the individual, the computer system may provide, addressed to an electronic device, a request for additional data (operation 214), where the additional data includes one or more of: an additional data instance, an additional type of data that is different from the types of data, or additional glaucoma diagnostic or glaucoma treatment data at one or more different sampling times than the sampling times of the data instances.

Alternatively, when the set data is determined to be medically meaningful (operation 212) for the individual, the computer system may: analyze the set of data to compute analysis results (operation 216); and selectively provides, addressed to the electronic device and/or a second electronic device, the feedback (operation 218) associated with glaucoma of the individual or glaucoma treatment of the individual, where the feedback is selectively provided based at least in part on analysis results. Note that the electronic device may be associated with the individual and the second electronic device may be associated with a clinician or a healthcare provider of the individual.

Moreover, the determining (operation 212) and/or the analyzing (operation 216) may be performed using a pretrained model. For example, the pretrained model may include a supervised-learning model or a neural network. In some embodiments, the determining (operation 212) and/or the analyzing (operation 216) may be based at least in part on one or more of: a glaucoma stage or risk of the individual (or a severity of the glaucoma); a prescribed medication used by the individual (e.g., for the glaucoma and/or one or more other medical conditions); one or more surgical, medical, or laser treatments or interventions performed on an individual; patient-supplied input (such as an adverse medication effect); a clinician-defined alert (or insight) criterion (such as a criterion for providing an alert or an insight); a relevant clinical insight or question;

and/or demographic information of the individual (such as an ethnicity or race, age or a socioeconomic group).

Furthermore, the feedback may include a treatment recommendation, such as a change to a current treatment of the individual.

In some embodiments of method 200, there may be additional or fewer operations. For example, in some embodiments, in addition to the determining (operation 212), the computer system may determine whether an existing (larger) set of data is medically meaningful. Furthermore, the order of the operations may be changed, and/or two or more operations may be combined into a single operation.

Embodiments of the monitoring techniques are further illustrated in FIG. 3, which presents a drawing illustrating an example of communication among components in monitoring device 110, electronic device 112 and computer system 130. In FIG. 3, one or more sensors 310 in monitoring device 110 may perform measurements 312. These measurements may be provided to a processor 314 in monitoring device 110.

Then, processor 314 may provide an instruction 316 to interface circuit (IC) 318 in monitoring device 110. In response, interface circuit 318 may provide one or more packets or frames 320 to electronic device 112 with information 322 specifying or corresponding to measurements 312.

After receiving the one or more packets or frames 320, an interface circuit 324 in electronic device 112 may provide information 322 to processor 326 in electronic device 112. Then, processor 326 may provide an instruction 328 to interface circuit 324. In response, interface circuit 324 may provide one or more packets or frames 330 to computer system 130 with information 322 specifying or corresponding to: measurements 312. Alternatively, in some embodiments, interface circuit 324 may provide the one or more packets or frames 320 or 330 to computer system 130 without providing information 322 to processor 326.

(While the preceding discussion and FIG. 3 illustrate communication between monitoring device 110 and computer system 130 occurring via electronic device 112, in other embodiments the communication between monitoring device 110 and computer system 130 occurs directly and, thus, without electronic device 112. For example, monitoring device 110 may communicate with computer system 130 using a cellular-telephone network and/or the Internet.)

Moreover, after receiving the one or more packets or frames 330, an interface circuit 332 in computer system 130 may provide information 322 to processor 334 in computer system 130. Then, processor 334 may optionally access additional measurements (AD) 336 in memory 338 in computer system 130, such as previous instances of measurements 312.

Next, processor 334 may assess 340 whether measurements 312 and the optional additional measurements 336 are medically meaningful. When measurements 312 and the optional additional measurements 336 are not medically meaningful, processor 334 may instruct 342 interface circuit 332 to provide a request 344 for additional or different data to monitoring device 110 and/or electronic device 112. Based at least in part on request 344, monitoring device 110, electronic device 112 and/or computer 130 may repeat some or all of the preceding operations (e.g., until a medically meaningful set of data is obtained).

Alternatively, when measurements 312 and the optional additional measurements 336 are medically meaningful, processor 334 may analyze 346 measurements 312 and the optional additional measurements 336, and may determine or select feedback 348 (such as a treatment recommendation). For example, processor 334 may access a pretrained analysis model in memory 338. This analysis model may use measurements 312 and the optional additional measurements 336 as inputs and may output results of assessment 340, analysis 346 and/or feedback 348.

Furthermore, processor 334 may store information 350 specifying results of analysis 346 and/or feedback 348 in memory 338. Alternatively or additionally, processor 334 may instruct 352 interface circuit 332 to provide one or more packets or frames 354 with information 350 to computer 132.

After receiving the one or more packets or frames 354, computer 132 may provide information 350. For example, computer 132 may generate a user interface based at least in part on information 350 and then may display the user interface on a display.

While FIG. 3 illustrates communication between components using unidirectional or bidirectional communication with lines having single arrows or double arrows, in general the communication in a given operation in this figure may involve unidirectional or bidirectional communication.

In some embodiments, the monitoring techniques provide an ophthalmic monitoring system with smart alerts. This ophthalmic monitoring system may include one or more ophthalmic devices (such as a monitoring device) that measure or generate: physiological data from a user; environmental data; device data; and/or user action data.

Moreover, components in the ophthalmic monitoring system may communicate via interface circuits using wired and/or wireless communication. Furthermore, the ophthalmic monitoring system may include one or more computing locations (such as a computer system) that: store and process system data from data sources; and manage relationships between patients and clinicians, such as treatments of the patients. For example, the ophthalmic monitoring system may: transmit and/or receive data to and/or from the one or more ophthalmic devices; transmit and/or receive data to and/or from one or more terminals where data is input or received by a user; and/or receive one or more clinician-defined alert conditions. Note that a given clinician-defined alert condition may be defined using a clinically significant endpoint, where the endpoint may use more than one data source. Furthermore, the ophthalmic monitoring system may analyze data by applying one or more mathematical functions to the data. An output from the one or more mathematical function(s) with regard to the data may trigger at least one of: no action; a patient interaction; and/or the meeting of a clinician-defined alert condition. When an alert condition is met, the ophthalmic monitoring system may communicate an alert to at least one of: a user terminal; and/or an ophthalmic device. Additionally, the one or more user terminals may: display data to a user; receive data input from a user; and/or communicate alerts to a user.

Note that the physiological data may relate to or include at least one of: intraocular pressure; retinal nerve fiber layer thickness; visual field function; optic nerve images; eye laterality; and/or ganglion cell complex thickness.

Moreover, the environmental data may relate to or may include at least one of: time; location; altitude; and/or an environmental condition (such as elevation, temperature or relative humidity).

Furthermore, the device data may include device logs (such as an error log).

Additionally, the patient action data may be generated by or measured using at least one of: an accelerometer; a gyroscope; a camera; a user interface; a medication sensor (which monitors consumption or use of a medications); and/or another type of sensor.

In some embodiments, the patient action data may indicate at least one of: administration of a medication; ordering or refilling a medication; taking an intraocular pressure measurement; performing a visual field test; obtaining an optic nerve photograph or image; and/or obtaining an optic nerve optical coherence tomography scan.

Moreover, the one or more mathematical function(s) applied to the data may be performed by the ophthalmic device and/or the user terminal.

Furthermore, the interface circuits may: determine whether communication with the computing location is available or not available; selectively transfer data to the computing location when the computing location is available; and/or otherwise, when the computing location is unavailable, attempt to reconnect and/or communicate a notification to a user.

Additionally, a clinician-defined alert condition may relate to or may include at least one of: intraocular pressure; treatment compliance; treatment response; treatment duration; retinal nerve fiber layer thickness; visual field function; and/or ganglion cell complex thickness. For example, the treatment response may relate to or may include: medication treatment (such as a prescribed medication); surgical treatment; and/or laser treatment. In some embodiments, the clinician defined alert condition may relate to or may include: mean intraocular pressure; peak intraocular pressure; a range of intraocular pressure; and/or a specified severity and/or timing of the alert.

Note that the computing location may include: a remote server computing location, a fog computing location (which is located between a data source and the cloud), and/or an edge computing location.

Moreover, a patient interaction may relate to or may include the prompting of an action for at least one of: remeasurement or reobtaining of data from an ophthalmic device; administration of a medication; input by a user into a terminal; and/or communication with a clinician. For example, the remeasurement or reobtaining of physiological data may be prompted for a different time of day or to acquire a measurement if the user did not perform a measurement yet on a given day. Alternatively or additionally, an administration of one or more medications may be prompted for a different time of day or to take one or more medications on a day where the user may have forgotten to take the one or more medications. In some embodiments, an input by a user into a terminal may include one or more of: medication side effects; medication cost; medication compliance; medication supply or refill; and/or device use.

Furthermore, a patient interaction may include the communication of information to a patient. For example, the communication of information to a patient may relate to or may include at least one of: medication compliance data; ophthalmic device data; and/or treatment efficacy or response.

Another embodiment provides a method for remote ophthalmic monitoring with smart alerts. This method may include assigning one or more ophthalmic devices to one or more: patients; clinicians; and/or treatments. Moreover, the method may include: inputting into a terminal one or more clinician-defined alert conditions; obtaining data via one or more ophthalmic device(s) and/or terminals; transferring of the data via wired or wireless communication to one or more remote computing location(s); and/or analyzing the data by applying one or more mathematical functions to the data, where an output from the one or more mathematical function(s) with regard to the data may determine at least one of: no action; a patient interaction; and/or the meeting of an alert condition. Furthermore, the method may include: communicating a patient interaction to a user at one or more terminal(s) when determined; and/or communicating an alert at one or more terminals when an alert condition is met. Note that a mathematical function may be applied to the data prior to transfer of data to the remote server.

Another embodiment provides an ophthalmic monitoring system with clinical insights. The ophthalmic monitoring system may include one or more ophthalmic devices capable of generating or measuring one or more of: physiological data from a user; environmental data; device data; and/or user action data. Moreover, the ophthalmic monitoring system may include interface circuits that communicate using wired and/or wireless communication. Furthermore, the ophthalmic monitoring system may include one or more computing locations, where the computing location(s) may: store and process system data from data sources; and manage relationships between patients and clinicians, such as treatments of the patients. For example, the ophthalmic monitoring system may: transmit and/or receive data to and/or from the one or more ophthalmic devices; transmit and/or receive data to and/or from one or more terminals where data is input or received by a user. Additionally, the ophthalmic monitoring system may analyze data by applying one or more mathematical functions to the data, where: an output from the one or more mathematical function(s) with regard to the data may determine at least one of: no action; a patient interaction; and/or a data insight that relates to a clinically significant insight derived from more than one data source. In addition, the ophthalmic monitoring system may include: one or more user terminals that: display data to a user; receive data input from a user; and/or communicate data insights to a user.

Note that the physiological data may relate to or may include at least one of: intraocular pressure; retinal nerve fiber layer thickness; visual field function; optic nerve images; eye laterality; and/or ganglion cell complex thickness.

Moreover, the environmental data may relate to or may include at least one of: time; location; altitude; and/or an environmental condition (such as elevation, temperature or relative humidity).

Furthermore, the device data may include device logs, such as an error log.

Additionally, the device data may include raw data that may be unrelated to physiological data.

In some embodiments, the patient action data may be measured or generated by at least one of: an accelerometer; a gyroscope; a camera; a user interface; a medication sensor; and/or another sensor.

Moreover, the patient action data may indicate at least one of: administration of a medication; ordering or refilling a medication; taking an intraocular pressure measurement; performing a visual field test; obtaining an optic nerve photograph or image; and/or obtaining an optic nerve optical coherence tomography scan.

Furthermore, the one or more mathematical function(s) applied to the data may be performed by the ophthalmic devices and/or a user terminal.

Additionally, the interface circuits may: determine whether communication with the computing location is available or not available; selectively transfer data to the computing location when the computing location is available; and/or otherwise, when the computing location is unavailable, attempt to reconnect and/or communicate a notification to a user.

Note that a computing location may be: a remote server computing location; a fog computing location; and/or an edge computing location.

In some embodiments, a patient interaction may relate to or may include the prompting for at least one of: remeasurement or reobtaining of data from an ophthalmic device; administration of a medication; input by a user into a terminal; and/or communication with a clinician. For example, the remeasurement or reobtaining of physiological data may be prompted for a different time of day or to acquire a measurement if the user did not perform a measurement yet on a given day. Alternatively or additionally, an administration of one or more medications may be prompted for a different time of day or to take one or more medications on a day where the user may have forgotten to take the one or more medications. In some embodiments, an input by a user into a terminal may include one or more of: medication side effects; medication cost; medication compliance; medication supply or refill; and/or device use.

Moreover, a patient interaction may include the communication of information to a patient. For example, the communication of information to a patient may relate to or may include at least one of: medication compliance data; ophthalmic device data; and/or treatment efficacy or response.

Furthermore, a data insight may relate to or may include at least one of: glaucoma disease progression, intraocular pressure; visual field function; retinal nerve fiber layer thickness; ganglion cell complex thickness; medication compliance; medication tolerability; medication response and/or effect; device accuracy; and/or device errors. For example, the data insight may include: an unsupervised machine learning insight; and/or a supervised machine learning insight.

Another embodiment provides a method for remote ophthalmic monitoring with clinical insights. This method may include assigning one or more ophthalmic devices to one or more: patients; clinicians; and/or information specifying treatments. Moreover, the method may include: obtaining data via one or more ophthalmic device(s) and/or terminals; transferring the data via wired and/or wireless communication to one or more remote computing location(s); analyzing the data by applying one or more mathematical functions to the data, where an output from the one or more mathematical function(s) with regard to the data determines at least one of: no action; a patient interaction; and/or a data insight; communicating a patient interaction to a user at one or more terminal(s) when determined; and/or communicating a data insight at one or more terminal(s) when determined. Note that the one or more mathematical function(s) may be applied prior to transfer of the data to the remote server.

We now further describe embodiments of the monitoring techniques. Glaucoma is a chronic progressive blinding disease that is often monitored by measuring intraocular pressure (IOP) and that may be treated by lowering intraocular pressure. Note that techniques for monitoring glaucoma and assessing a risk of vision loss may include: assessment of visual field function via a visual field analyzer or perimeter, an optic nerve exam, a retinal nerve fiber layer and ganglion cell complex thickness obtained via OCT, a central corneal thickness, corneal hysteresis, and/or gonioscopy. Consequently, one or more testing modalities may be used in disease management. However, presently the only modifiable risk factor in the treatment of glaucoma is intraocular pressure. Intraocular pressure may be lowered via medications, lasers, or surgical intervention. Because intraocular pressure plays such an important role in both the treatment and monitoring of glaucoma, accurately measuring intraocular pressure both before and after treatments is typically important in order to understand risk and to assess the effect of treatment.

Usually, intraocular pressure is measured using a monitoring device referred to as a ‘tonometer.’ Notably, Goldman Applanation Tonometry (GAT) remains the reference-standard tonometry technique and involves measuring the force required to flatten the cornea with a prism of known surface area.

Traditionally, tonometry is performed in office every three months or less in a glaucoma patient, with subsequent treatment of high pressures to a certain threshold as determined by the clinician. Clinicians often establish a goal pressure for which it is ideal for the IOP to stay below. Such an IOP goal is usually unique to each patient. A patient with mild stage glaucoma may have a higher IOP goal than a patient with severe stage glaucoma. Typically, staging of a glaucoma patient may depend on loss of visual field function as determined by a visual field test. Thus, a patient with more-advanced field loss often may require a lower IOP treatment goal.

Note that advancement of treatment in an attempt to lower eye pressure further often may be determined by whether a patient is below or above their IOP goal at subsequent office visits. Complicating this analysis is the diurnal nature of IOP. While tonometry in the office obtains IOP at a single discrete point in time, in reality, intraocular pressure has diurnal fluctuation with troughs and peaks. Studies have shown more than 50-75% of IOP peaks occur outside of clinic hours. Consequently, a single IOP measurement by tonometry in a clinic may not accurately represent a patient's true IOP profile in relation to their goal.

For example, a single low IOP measured in clinic may lead a clinician to assume a patient's IOP is well controlled, when in reality the IOP may be much higher at other times during the day. In such a case, the patient may continue to lose vision despite seemingly controlled in-office IOP measurements. Home-based tonometry, in which a patient can measure their IOP at home, or continuous tonometry from implantable or one or more contact-lens integrated sensors can be helpful to indicate the diurnal fluctuation of IOP.

Another complication in the management and treatment of glaucoma patients is medication compliance. Notably, medication eyedrop non-compliance is common among glaucoma patients. When a clinician begins a patient on a new IOP-lowering medication, a follow-up IOP measurement often tells the patient whether the treatment is effective or not. However, the measurement in a non-compliant patient may not represent a true effect of treatment. Even when the patient reports compliance with a new medication, it may be difficult to assess the etiology of a poor IOP response because the patient may: be non-compliant or forgetful in reality; have true non-response to the specific medication; and/or an IOP that is higher on the diurnal curve based at least in part on time of measurement.

In order to address these challenges, monitoring techniques that process remotely captured diverse data to provide requests to further improve the data, detect a clinician-defined alert condition, and/or produce a clinically useful data insight are described. The monitoring techniques may be implemented by one or more components that may be included in a system, such as: ophthalmic (or monitoring) devices, terminals, and/or computing locations.

Note that users of the one or more components may include: patients, clinicians, and manufacturers. Moreover, ophthalmic devices may measure or generate physiological, environmental, device, and/or patient-action data sets. In some embodiments, physiological data may include data that is obtained by an ophthalmic device to determine IOP. However, in other embodiments, physiological data may include visual field data from a visual field analyzer, retinal nerve fiber layer or ganglion cell complex data obtained by an OCT machine, or optic nerve head photographs or images. Furthermore, physiological data may represent eye laterality, such as a right or a left eye.

Additionally, environmental data may include parameters related to the environment of the ophthalmic device, such as: a time of measurement, a location, an altitude or an elevation, relative humidity, and/or temperature. For example, altitude or elevation may be measured using a pressor sensor or using the Global Positioning System. Device data may include data that relates to the function or physical state of the device itself, such as: an active assignment to a specific patient identifier, tamper sensors, error logs, and/or non-physiological data related to the device at the time of measurement (e.g., device tilt or misuse). Moreover, patient-action data may include data recorded related to an action by the patient, such as: a successful IOP measurement, an unsuccessful attempt at taking an IOP measurement, or failure to complete the entirety of a visual field test.

In some embodiments, an ophthalmic device may not be able to capture physiological data, but may be able to capture environmental, device, and/or patient action data. For example, the ophthalmic device may include a medication compliance monitoring device that captures data about when a patient administers a medicated eyedrop, a time of administration, and/or a type of medication. Moreover, a given ophthalmic device may be capable of transmitting the data.

Terminals may include any location where data is input or received by a user in the system, such as: a desktop computer, a smartphone, a tablet, etc. Furthermore, computing locations may include any location where data is stored and processed, such as a cloud computing platform or a remote server.

During operation, components in the system may exchange data, process multiple variables and communicate patient interactions. These capabilities may allow one or more of the components to obtain additional data that can be used to determine useful insights or when a clinician-defined alert condition is met, while avoiding unnecessary alerts or misinforming insights.

As described previously in FIG. 1, a typical data flow may start with a clinician using computer 132 deciding that remote monitoring of a patient's glaucoma is necessary. The clinician may enter initial data (or a setup) related to the patient's medical condition along with one or more alert conditions, as appropriate, based at least in part on the medical history of the patient. In response, computer 132 may provide this information to computer system 130. Note that the initial setup data may include: a patient identifier, a stage or risk of disease, medications or treatments and their start or modification dates, etc. In some embodiments, a specific alert condition may not be provided by the clinician.

Moreover, the clinician may instruct the patient to take home one or more ophthalmic devices (such as monitoring device 110) for remote monitoring and treatment of their disease. As the patient uses monitoring device 110 at home, monitoring device 110 may wirelessly transmit (via electronic device 112) the data it collects to computer system 130, where it is stored in memory along with the patient, clinician, and clinic setup data. Furthermore, as described further below, additional data may be entered by the clinician and/or patient via a terminal (such as computer 132 or electronic device 112) when a patient interaction occurs (such as a patient interaction associated with a clinician-defined alert condition).

Additionally, computer system 130 may perform assessment and/or analysis of sets of data for the purpose of selectively triggering an alert (when applicable) or communicating a request, such prompting an action by a clinician or a patient. Patient interactions (such as a request to obtain additional data) may help clarify an insight or whether an alert condition is met before the insight or alert is communicated. This functionality may reduce unnecessary or erroneous alerts that otherwise would occur in glaucoma monitoring. For example, computer system 130 may prompt an individual to perform additional measurements using an ophthalmic device when a set of data is not medically meaningful.

Once appropriate data has been collected and analysis (such as one or more mathematical functions that are applied to the data) have determined an alert condition has been met, computer system 130 may communicate an alert to a clinician per the conditions they input during the initial setup for monitoring the patient. Moreover, after the clinician receives an alert or a message, they may log into a terminal (such as computer 132) to review the data and insights generated by computer system 130, and they may use this information as an input in their decision-making process in providing care for the patient.

As noted previously, computer system 130 may allow a clinician to define and input one or more alert conditions for which they would like to receive messages or notifications (which are sometimes referred to as ‘alerts’) when computer system 130 determines that an alert condition has been met. In this way, computer system 130 may provide accurate and scalable monitoring of the patient's disease while avoiding or excluding non-useful or incorrect alerts. The alert conditions may be determined based at least in part on a clinically significant endpoint that corresponds to more than one data source. For example, a clinically significant endpoint may include: a mean IOP threshold, loss of retinal nerve fiber layer, progression of visual field loss, or lack of therapeutic response of a medication, and/or another endpoint. By including more than one data source, the clinically significant endpoint may be met meaningfully, rather than simply technically. This capability may reduce a false alarm rate, because communicating an alert when an alert threshold is technically met based on one data point or source may not be useful and may lead to too many alerts and/or clinician alert fatigue. Thus, computer system 130 may use data collected from multiple relevant sources to determine if the alert condition has been meaningfully met before communicating an alert to the clinician. Examples of medically meaningful combinations of data are described further below.

Note that when computer system 130 does not have enough data to make a decision on the meeting of an alert condition, it may provide a patient interaction or a request that prompts a patient to obtain more data to clarify the dataset before computer system 130 determines whether the alert condition has been met. Thus, patient interaction may be unique and integral to the monitoring techniques.

While some alert conditions may be more serious than others, and thus may require more urgent review or intervention, a clinician may opt to characterize each alert condition different and/or may define the timing of communication of an alert. For example, one alert condition may be characterized by a clinician as a ‘soft’ or non-urgent alert, while another may be defined as a ‘hard’ or urgent alert.

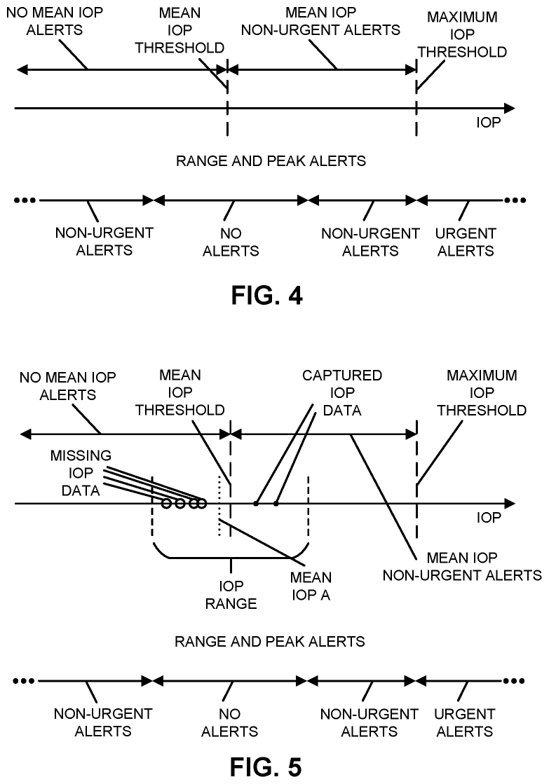

In some embodiments, monitoring device 110 may include an ophthalmic device that is used to measure IOP. Clinically relevant parameters in a resulting set of data may include: a mean IOP, a peak IOP, and/or a range of IOP values. A clinician may input a non-urgent alert condition requesting notification when the mean IOP value is greater than 16 mm Hg or outside a range of 12 to 20 mm Hg. Because these events may be characterized as non-urgent, the clinician may define that they want the alert only communicated when they open the patient's chart for monthly review of data. Additionally, the clinician may designate a hard alert, such as: a peak IOP of 28 mm Hg. When the peak IOP is greater than this threshold value an urgent alert may be communicated to the clinician, e.g., a real-time push notification to the clinician's cellular telephone or email. This is illustrated in FIG. 4, which presents an example of IOP measurements and threshold values.

Notably, when a patient takes IOP measurements with values that fall outside the alert condition thresholds, an alert may not be communicated. For example, when the alert condition includes a mean IOP threshold, and only one or two measurements are greater than this threshold, the mean IOP alert condition may be technically met, but may not be useful for a clinician because there may not be sufficient data points to reach a meaningful conclusion. Indeed, if more measurements were acquired, the IOP values may be lower given the diurnal and fluctuating nature of IOP. Consequently, mean IOP value A may actually be below the alert threshold. This is illustrated in FIG. 5, which presents an example of IOP measurements and threshold values.

In addition to computer system 130 processing the data to reach meaningful conclusions, it may also prompt or request action by the patient to obtain a variety of additional data, thereby capturing more variables and improving the dataset quality to reach better conclusions. For example, in the preceding embodiments, computer system 130 may wait for additional measurements to improve the dataset prior to determining an alert is needed and then presenting or providing the alert, but if the patient stopped at only two measurements, computer system 130 may prompt the patient to take additional measurements until a meaningful conclusion is obtained.

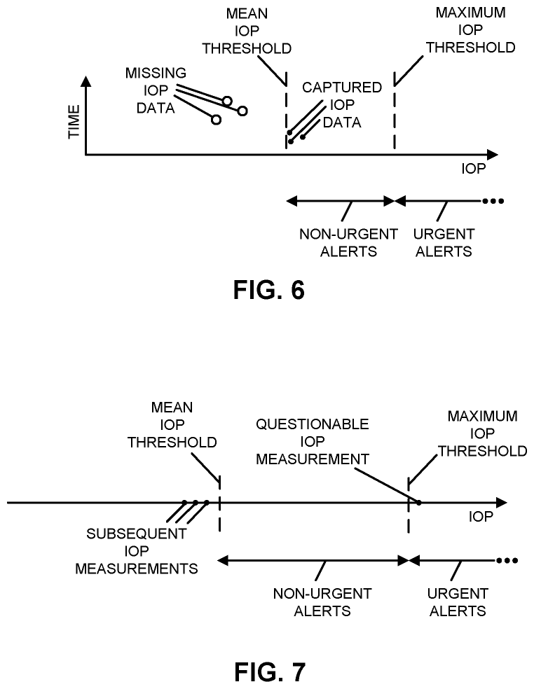

In another example, another data source may be included in the analysis, such as environmental data (e.g., the time of measurement). As described previously, IOP has diurnal fluctuation with toughs and peaks, and tends to be higher in the early morning hours. Thus, as shown in FIG. 6, which presents an example of IOP measurements as a function of time and threshold values, the clinician may define a mean IOP non-urgent alert threshold condition and a maximum urgent alert condition. However, in this example, all the data points obtained were in the early morning hours, e.g., by a patient who tends to perform IOP measurements when they first get out of bed. Consequently, while the captured IOP readings are higher than the alert threshold, because they are consistently in the early morning the patient likely has much lower IOP values later in the day. Thus, computer system 130 may determine that the data needs to be improved before determining whether the alert condition is met in order for an eventual alert to be useful. In this case, computer system 130 may wait for additional data sampling times, or request that the patient record IOP measurements at other times in the day in order to improve the dataset before determining whether the alert condition has been met. Therefore, including the time of measurement in this case may improve the data and may be used to guide subsequent action(s) by the patient or clinician.

Another example illustrates the use of device data. In this case, a patient may have taken an IOP measurement that is in an ‘urgent’ zone, which specifies an absolute threshold for an urgent alert to the clinician. However, the device error log data may indicate that the IOP measurement is of questionable reliability, e.g., because the patient blinked at the time of measurement. Consequently, the error log data may nullify the previous data point and computer system 130 may prompt the user to take another IOP measurement. The subsequent IOP measurement may be below the threshold and the subsequent error log data may indicate that this is a reliable measurement. This is illustrated in FIG. 7, which presents an example of IOP measurements and threshold values.

In some embodiments, monitoring device 110 or an ophthalmic device (e.g., with a drop sensor, and which may or may not be an electronic device) may include an ophthalmic device that measures or generates IOP data from physiological data, and electronic device 112 may determine or generate patient action data (such as medication-compliance monitoring) to capture when the patient performs the action of taking an IOP lowering eyedrop for the treatment of glaucoma. Monitoring device 110 and electronic device 112 may each send or communicate environmental and device data. In this example, monitoring device 110 and electronic device 112 may be linked to or associated with the same patient identifier and the same clinician. In addition to alert thresholds, computer system 130 may receive and/or manage relationships regarding treatment information specific to the patient. For example, the clinician or patient may provide an input to computer system 130 that indicates that the patient has been prescribed a medication X, which is supposed to be taken twice a day. Further details about medication X may also be input to or may already be defined in computer system 130, such as a duration of action of medication X.

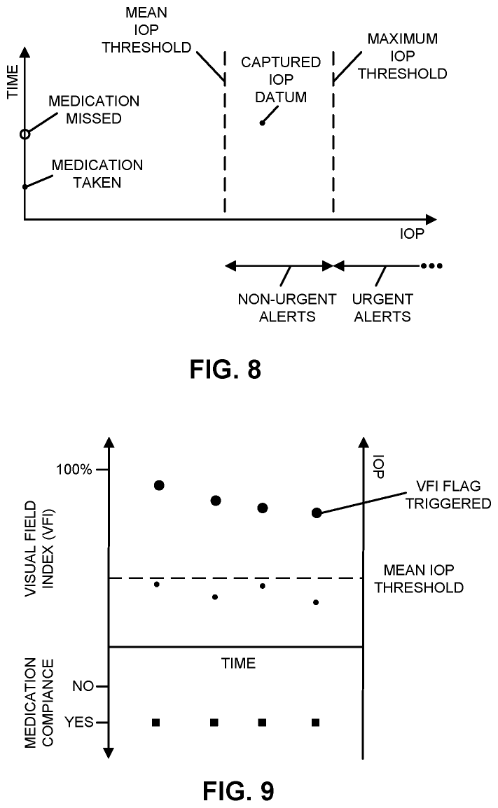

With these additional parameters (such as the time of a medication is taken, a type of medication, etc.), the data and alert/action decision tree may be more complicated. For example, consider a patient who has been prescribed medication X, which is supposed to be taken twice a day with a 12-hour duration of action. This data may be input to computer system 130 and the clinician may have defined a mean IOP threshold alert condition. The patient may subsequently check their IOP value using an ophthalmic device in the evening hours, and the IOP may be greater than the threshold. Moreover, the patient may typically be compliant in taking their morning eyedrop dose (as recorded by electronic device 112), but the patient may tend to forget to take their evening dose. This is illustrated in FIG. 8, which presents an example of IOP measurements as a function of time and threshold values.

In this case, computer system 130 may provide an actions prompt and a clinical alert in a useful manner to improve the care of the patient, and to assist the clinician with decision making. Notably, when the evening drop is missed, computer system 130 may label the IOP value that is greater than the threshold as an ‘untreated IOP’ datum because the evening eyedrop was not taken. In general, the interaction between IOP and a specific medication with a specific duration of action, taken at a specific time of dosing in relation to a time of IOP measurement, may significantly increase the complexity of relationships among data. Consequently, a number of labels or types of metadata may be generated. In this simplified example, the non-compliant or ‘untreated IOP’ datum may not be considered to meet a meaningful alert condition. In an effort to better treat the patient and to improve the therapeutic response data, computer system 130 may provide a request to the patient suggesting or reminding the patient to take their evening eyedrop (thereby allowing computer system 130 to obtain ‘treated IOP’ data). In the event of a successful evening drop placement, a repeat ‘treated IOP’ datum may be counted towards meeting the alert condition.

In some embodiments, a clinician-defined alert condition and multiple ophthalmic devices may be used. Notably, a first ophthalmic device may collect IOP data, a second ophthalmic device may collect medication compliance data, and a third ophthalmic device may collect visual field or perimetry data. In this example, the clinician may want to be alerted if the visual field index (a parameter on a visual field analyzer that describes visual function) decreases in relation to IOP treatment threshold goal. As discussed in the previous example, the patient may take an IOP lowering eyedrop twice a day, but may also perform home or out-of-office IOP measurements, as well as an occasional home or in-office visual field test, on a visual field analyzer. As shown in FIG. 9, which presents an example of visual field index measurements (represented by the large filled circles), IOP measurements (represented by the small filled circles) and medication compliance (represented by the filled squares) as a function of time, the patient may be compliant in taking the eyedrop medication and the measured IOP values may be less than the treatment IOP goal. However, in this case, the visual field tests may indicate a worsening of the visual field index. In response, computer system 130 may determine an alert to the clinician that the visual field is worsening despite compliant treatment and IOP goals that are within the physician's treatment threshold. This information may help the clinician determine the best next action, such as advancing the patient's treatment by adding another IOP lowering medication to the patient's regimen and/or lowering the IOP threshold.

As discussed previously, medication non-compliance and the resulting data labeled ‘untreated IOP’ may be used by computer system 130 to provide a clinical insight to the clinician, even if the clinician has not requested such data using a predefined alert condition. Because a variety of clinical insights may be determined from the data, it may be cumbersome for the clinician to provide defined alert conditions for every possible scenario. Instead, by analyzing the data from different sources, computer system 130 may provide clinical insights that can help the clinician to more accurately tailor treatment decisions to a given patient. For example, a prescribed medication is typically only useful if the patient is compliant in taking the medication. Thus, in the case of untreated IOP, an insight may include a notification to the clinician that eyedrop doses are frequently missed and that the resulting untreated IOP data is greater than the defined alert threshold. This insight may be clinically useful. Alternatively, when non-compliant IOP data is consistently greater than an alert threshold such that the clinician may feel further vision loss may occur, this information may help the clinician to: further investigate the reasons for non-compliance; pursue options to improve compliance; or change the treatment.

Expanding on this example, suppose the trend of medication non-compliance with subsequent IOP data greater than threshold in the evening hours continues and computer system 130 provide this insight to the clinician. This may lead to the clinician to ask the patient why evening doses are frequently missed. This patient interaction may take place via patient and clinician terminals, such as electronic device 112 and computer 132. In this scenario, the patient may communicate to the clinician that the second dose of drug Y is causing significant ocular irritation which is why the patient does not take it. In response, the clinician may decide to change the prescribed medication. Alternatively, the clinician may decide to stop the medication and to perform a treatment that lessens the risk of non-compliance and ocular toxicity, such as an implanted medication, or a laser or surgical procedure to reduce the IOP.

In another example of medication non-response, a clinician may prescribe a medication that the patient is compliant in taking. In a traditional clinical setting, it is often difficult to determine if an IOP lowering medication has significantly lowered a patient's IOP given the complexity of or interactions between the large number of variables. While medication response may not be defined as an alert condition, it may be a useful insight for the clinician. Consequently, computer system 130 may clarify medication response based at least in part on the interaction of IOP measurements, medication specifics, medication compliance, a treatment start date, and/or treatment times.

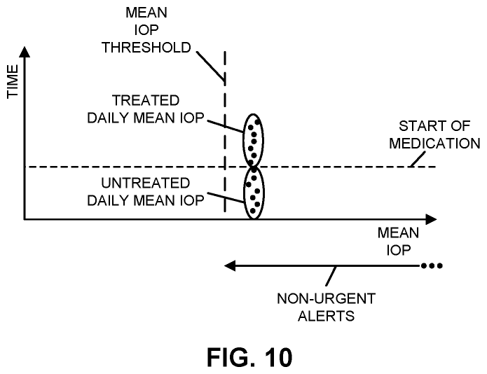

For example, a patient may have a pre-treatment mean IOP of 18 mm Hg, but an IOP mean threshold of 16 mm Hg as determined by the clinician. The clinician may then prescribe medication Y. As shown in FIG. 10, which presents an example of IOP measurements as a function of time and threshold values, a patient may start using the medication on February 1, after January IOP measurements determine a mean IOP of 18 mm Hg. Subsequent measurements obtained in February may determine a mean IOP of 18 mm Hg using appropriate data sources as previously described. Thus, the mean IOP may still be meaningfully greater than the threshold. In a traditional in-office clinical scenario, it may be difficult for the clinician to determine the cause of a lack of response from a single IOP reading and the lack of compliance data. Diurnal IOP variation in relation to the in-clinic time of measurement or a suspicion of non-compliance may mislead the clinician to believe that there may be a treatment affect, when there really isn't one. The disclosed monitoring techniques may confirm the therapeutic non-response using a system-derived insight, which may be communicated by computer system 130 to the clinician and which may lead the clinician to change therapy. While this is a simplified scenario, in general a number of additional variables could be included in the analysis in addition to the determined medication response.

Another example of a system-derived insight may include the ability to assess the efficacy of a drug implant, as well as the duration of effect. Similar to the preceding example, there may be a pre-treatment mean IOP of 18 mm Hg in January, after which a drug implant is surgically placed into the patient's eye. Subsequent IOP measurements may be performed in February with prompting to obtain diurnal data, which may be medically meaningful and may indicate a meaningful decrease in the mean IOP. In March, an increase in the mean IOP may be determined by computer system 130, which may indicate a possible decrease in drug effect. When presented to the clinician, this insight may lead the clinician to either re-dose the implant or to add a different medication depending on their treatment goals.

Note that it may be difficult or impossible for the clinician to determine the described clinical insights by reviewing disparate data sources, questioning a patient for further information (or action), combining all data together, etc. This is because of problems such as patient memory, difficulties in combining disparate data sources, time constraints on the physician, missing data, and other confounding factors. The ability of computer system 130 to relate the disparate data, to prompt the user for information and/or for action when needed, to record relevant inputs and outputs, and then to determine useful clinical insights may assist clinicians in providing care to an expanding patient population under cost constraints.

In some embodiments, the monitoring techniques may provide clinician-defined alert conditions. Notably, computer system 130 may allow a clinician to input an alert condition unique to the management of glaucoma, as well as to define the severity and/or timeframe of a notification when the alert condition is met. By including multiple data instances, types of data, and/or sampling times, the system may ensure that the alert criteria may be meaningfully met (as opposed to just technically met) prior to presentation of the alert to the clinician. Additionally, a characterization of the severity or timeframe of notification may help avoid notification fatigue for non-urgent alert conditions that are met.

Moreover, in addition to one or more alert criteria, smart alerts may include answering clinical questions that are asked by a clinician by: recruiting/gathering of appropriate data sources, data types, and/or timeframes. By allowing a clinician to ask questions in a similar manner to how they may do so in a clinic, and then providing an answer to the clinician, the monitoring techniques may make it less likely that the clinician discontinues using computer system 130.

For example, the monitoring techniques may use severity-weighted peak IOP. Notably, in current medical practice, a clinician may have a patient with pre-perimetric or mild glaucoma that has been prescribed an IOP-lowering medication. Based at least in part on the patient's mild stage, the clinician may have established an IOP goal of 18 mm Hg. In current medical practice, this may mean that at each visit that the clinician sees the patient and measures the IOP in-office (e.g., every 3-4 months), the IOP should be 18 mm Hg or below. If the IOP is greater than 18 mm Hg on one or more occasion, or if there is clear progression of vision loss with in-office IOPs of 18 mm Hg or less, the clinician may decide to add another IOP-lowering medication, and to further lower the IOP goal. Thus, in existing medical practice, current IOP goals are typically defined as peak IOP goals.

As discussed previously, in-office IOP data often misses diurnal fluctuation of IOP. In contrast, home monitoring captures IOP values outside of clinic hours and may establish a more-accurate IOP profile. This capability may allow computer system 130 to use a severity-weighted peak-IOP alert criterion. Notably, when a clinician sets an alert condition of ‘IOP above 18 mm Hg’ in the preceding scenario, but now in the context of at home monitoring, the home measurements of IOP may fluctuate between 14 and 19 mm Hg, and the alert criterion may be met multiple times, so that there could be multiple notifications.

However, for this mild glaucoma patient, an IOP of 19 mm Hg may not be an urgent concern. Therefore, in current medical practice, an IOP slightly greater than the IOP goal at clinic visits may be noted, but may not be acted upon unless drastically higher or when there is progression of vision loss. This is because there is usually a risk/benefit balance to adding additional medications or to performing surgery to lower the IOP further. Therefore, the clinician may decide to monitor and recheck the IOP again at the next visit, and it may be many months before the patient's IOP is remeasured in the clinician's office again. Thus, multiple alert notifications in this scenario may cause alert fatigue, especially given that a glaucoma specialist may care for hundreds to thousands of glaucoma patients at one time.

In order to address this problem, computer system 130 may define this particular alert condition to integrate home monitoring into how clinicians practice medicine. Notably, the alert criteria may be to provide an alert when the IOP is greater than 18 mm Hg and the glaucoma severity low. However, because the severity is low, the alert may be designated as non-urgent and may not be presented until a monthly review of IOP data by clinician (or the next time the patient's chart is opened).

Furthermore, other IOP parameters may be more important than the peak IOP, such as: the mean IOP and a range of IOP values. In the same patient scenario described previously, the clinician may define the alert condition as a mean IOP greater than 18 mm Hg. This may avoid unnecessary alerts for IOP values that are greater than 18 mm Hg. However, given the diurnal nature of IOP, computer system 130 may determine when the IOP data accurately represents the reality of a patient's IOP before determining the mean IOP and any subsequent alerts. For example, IOP tends to be higher in early morning hours. Consequently, if two IOP measurements taken in the morning are 19 mm Hg, technically, the mean IOP is 19 mm Hg. Nonetheless, the IOP may be 13 mm Hg at other times of the day, so the true mean IOP may be less than 18 mm Hg. Therefore, alerts based simply on the mean IOP may not be useful for the clinician (or may not be medically meaningful).

Thus, computer system 130 may improve the alert criteria to take into account IOP measurements at other times of the day prior to alerting the clinician. For example, the alert criteria may be to provide an alert if the mean IOP is greater than 18 mm Hg and the glaucoma severity is low. However, the alert may not be presented until a monthly review of IOP data by clinician (or the next time the patient's chart is opened). When the patient only takes two IOP measurements and they are 19 mm Hg in early morning hours, computer system 130 may wait to see what further measurements show given that the clinician has designated the glaucoma severity and/or timeframe for notification. Alternatively, computer system 130 may request that the patient to take IOP measurements at other times of the day.

In the preceding two examples, defining the timing of an alert and how computer system 130 processes this set of data based at least in part on the alert-criteria parameters and relevant data types may make a subsequent alert more medically meaningful.

In some embodiments, severity-weighted peak IOP may be used for urgent conditions. Notably, patients with very severe glaucoma may have minimal residual vision, and a drastic rise in the IOP value may remove the remainder of their vision. Many of these patients have invasive surgical procedures to lower their IOP values by creating new drainage pathways that allow the aqueous fluid that fills the eye to drain out of the eye. However, in the post-operative period, some of these drainage pathways may scar up, and their IOP can rise drastically. Missing such an IOP spike is a common cause of vision loss in the post-operative period. Similarly, patients with a glaucoma subtype called pseudoexfolation glaucoma can be relatively controlled, and then may have a sudden severe rise of IOP. Thus, for some severe glaucoma patients, urgent notification of the peak IOP may be warranted. In such a patient, the IOP goal may be 12 mm Hg, but the clinician may want to know if the IOP value ever exceeds a peak of 16 mm Hg. In this case, the alert criteria may be to provide an alert if the IOP is greater than 16 mm Hg and the glaucoma severity is high and/or the clinician has designated an urgent timeframe for notification. Computer system 130 may send the alert notification immediately.

In another example, low IOP values may be used to trigger an alert. Notably, some incisional glaucoma surgeries use a tube from inside the eye to under the outside white part of the eye (the conjunctiva) to lower eye pressure. During the surgery, the tube is often tied with a dissolvable suture to stop early flow and to allow some scar tissue to form a backstop to flow. However, after 4-6 weeks, the suture will break and the IOP will lower as the tube begins to flow. It is often important for the clinician to know when this happens. Instructing the patient to regularly check IOP values with an alert criterion that specifies a significant drop from baseline in the post-operative period can be used to detect opening of the tube. In addition, a second alert criterion may be used with a high IOP value, such as when the tube has not opened yet because there is risk that the IOP may become too high. For example, the alert criteria for post-operative tube opening may include: a peak IOP greater than 25 mm Hg, glaucoma severity high and an alert timeframe instructing computer system 130 to provide the alert the next day; lowest IOP less than 10 mm Hg, glaucoma severity high, and an alert timeframe instructing computer system 130 to provide the alert the next day after confirmation with a second IOP measurement; and a drop in the mean IOP of greater than 20%, a glaucoma severity of high, and an alert timeframe instructing computer system 130 to provide the alert the next day.

In some embodiments, the monitoring techniques may provide broad clinical insight for progression. Notably, one of the big picture questions in the management of glaucoma is whether or not the patient's disease progressing. Attempts to answer this question typically take into account many data points from different data sources. Traditionally, a clinician may look at a patient's visual field test compared with the results of a prior test. For example, the clinician may determine: whether there are more spots where the patient is missing the light; and/or whether the optic nerve thickness is thinning on the OCT scans while the visual field progresses. Computer system 130 may analyze multiple data sources in this set of data together, including that the IOP is higher than in the previous year, and that the OCT nerve thinning corresponds to a possible area where more of the visual field is lost. Thus, computer system 130 may provide an alert or may provide insight indicating that there is higher likelihood that the patient is progressing.

More generally, how does computer system 130 determine whether there is disease progression? Indeed, what is disease progression? Nerve tissue may thin prior to any visual field loss noted on perimetry testing. Consequently, both of these events are considered progression of the disease. Sometimes the current dataset may not allow a clear determination to be made (at least, not yet). When this occurs, computer system 130 may assist the clinician by requesting more data to answer questions regarding the current set of data. For example, because the IOP has been higher than normal, and because during this time interval there may be nerve thinning in a current OCT scan relative to a previous OCT scan, computer system 130 may recommend another OCT scan to help confirm whether there has been progression.

In another example for pre-perimetric glaucoma (i.e., for patients with thinning of the optic nerve tissue on the OCT scan but NO visual field loss yet), clinicians may want to know when patients cross over from just tissue loss to loss of vision on the visual field test. In this case, a useful insight may include recommending a visual field test because the nerve fiber layer thickness has decreased in a current OCT scan relative to a previous OCT scan, and IOP values measured at home have increased. In response, a clinician may obtain another visual field test to check if new vision loss is present.

In some embodiments, an insight may be associated with the effect of medication. Notably, when a patient starts a new medication, a clinician typically wants to know how effective it is. Notably, studies have shown that there is reduced chance of glaucoma progression when IOP is lowered by 20-30%. Thus, computer system 130 may provide an alert in the form of a clinical question, “How effective is this medication?”

In addition to information specifying a medication, computer system 130 may use a start data of a medication (which may be provided by a clinician) when comparing current and previous mean IOP values. Because some medications may take, e.g., four weeks to reach full effect, while other medications may take full effect within 6 weeks, computer system 130 may assess the medication effect based at least in part on these medication-dependent timescales (which may indicate when IOP data is medically relevant). Thus, computer system 130 may only determine whether it has sufficient data to provide an alert when the mean IOP has stabilized. If the IOP does not change after an appropriate time interval, computer system 130 may provide an alert or a message indicating that the IOP has not been reduced, and that either the patient is non-compliant or the medication is ineffective. Moreover, when there is data indicating that the patient is compliant (and, thus, is taking the medication), computer system 130 may know when the IOP is expected to stabilize. This knowledge may allow computer system 130 to determine when to provide an alert based at least in part on therapeutic response or mean IOP threshold(s), and/or when to prompt a patient to take their medication before alerting the clinician.

In some embodiments, a clinician may provide a clinical question that is unique to a patient and/or alert criteria to computer system 130. When computer system 130 has appropriate (medically meaningful) data, computer system 130 may provide an answer to the clinical question by providing an alert. Note that the appropriate data may include data source(s) that comply with a measurement timeframe specified in the alert criteria and/or the alert may be provided based at least in part on an alert timeframe specified in the alert criteria.

Moreover, in some embodiments, the monitoring techniques may attempt to follow the clinical flow and thought process of a clinician. For example, when a new patient is seen, a clinician may: examine the patient; review their OCT scans, visual fields, and risk factors; determine whether the patient has glaucoma; and/or instruct the patient to purchase an ophthalmic device and to measure their IOP at home.

Furthermore, in some embodiments, IOP data may be considered medically meaningful when it captures the full scope of diurnal IOP, such as IOP measurements at different time of day. Consequently, when a patient only performs IOP measurements in the evenings, computer system 130 may wait (e.g., for up to a month) until morning and midday data is acquired and/or may prompt the patient to obtain this data.

When the IOP data is complete, the clinician may review the data and then may prescribe a medication (e.g., latanoprost) for use in both eyes on July 1. Moreover, the clinician may provide alert criteria, such as: provide an alert when the mean IOP is greater than 18 mm Hg, the glaucoma severity is low, and on a timeframe of when the patient chart is opened again. Furthermore, the clinician may define or select a clinical question alert: How well does this medication work?

After six week (a typical timescale for latanoprost to reach full effect), computer system 130 may determine whether diurnal IOP has stabilized at a lower level. If yes, computer system 130 may provide an alert to the clinician that latanoprost dropped the mean IOP 22% from the pre-treatment mean IOP. After reviewing the alert and the patient data, the clinician may be satisfied with the treatment response and may continue therapy and follow up with the patient in six months for an updated OCT scan. Consequently, the clinician may define or select alert criteria of providing an alert when the mean IOP is greater than 18 mm Hg, the glaucoma severity is low, and on an alert timeframe of when the patient chart is opened again or after one month (whichever is sooner).

When the patient returns for a follow-up appointment in six months for an OCT scan, the OCT scan results may be provided to computer system 130. In response, computer system 130 may provide an insight. Notably, computer system 130 may note that there has been a decrease in the nerve fiber layer thickness compared to the baseline OCT scan, and may recommend that another OCT scan be performed to confirm these changes. In response, the clinician may recommend patient follow-up in four months for another OCT scan. Moreover, the clinician may define or select alert criteria of providing an alert when the mean IOP is greater than 18 mm Hg, the glaucoma severity is low, and on an alert timeframe of when the patient chart is opened again or after one month (whichever is sooner). Furthermore, the clinician may specify a clinical question alert: Is the retinal nerve fiber layer progressing?

Furthermore, when the patient returns for the subsequent OCT scan, computer system 130 may provide an insight that the OCT scan confirms likely progression of thinning of nerve tissue at the inferior pole of the optic nerve. The clinician may review the test results and the insight, and may decide to proceed with a selective laser trabeculoplasty (SLT) to lower the patient's IOP. In response, computer system 130 may prompt the clinician to reset the most-recent OCT scan as a new baseline because a new treatment is being initiated (so that subsequent tests may be compared with the new baseline). Additionally, the clinician may instruct the patient to continue to monitor IOP at home and may define or select alert criteria of providing an alert when the peak IOP is greater than 25 mm Hg, and on an alert timeframe of the next day after confirmation with a second IOP measurement (which may be requested from the patient). Note that the clinician may specify a clinical question alert: How effective was the SLT?

Computer system 130 may detect a 15% reduction in mean IOP that plateaus at six weeks. The clinician may review these analysis results and may be satisfied with the progress. Consequently, the clinician may instruct the patient to continue to monitor IOP at home and to follow up in four months with a visual field test. Moreover, the clinician may lower the patient's IOP goal and define or select alert criteria of providing an alert when the mean IOP is greater than 14 mm Hg, the glaucoma severity is low, and on an alert timeframe of when the patient chart is opened again. Furthermore, the clinician may specify a clinical question alert: Is the visual field loss progressing?

After the patient follows up for the subsequent visual field testing, computer system 130 may provide an insight that: the visual field progressed in the superior nasal quadrant from the baseline field, which corresponds to continued decrease in nerve fiber layer thickness on OCT scan; two interventions have taken place since the comparison baseline visual field, with a mean IOP goal of 18 mm Hg or lower after starting latanoprost, and a mean IOP goal of 14 mm Hg or lower after SLT. Based at least in part on these insights, the clinician may want to watch the patient closely, because visual field damage may have occurred while the mean IOP was 16 mm Hg, below the first goal of 18 mm Hg but above the new goal of 14 mm Hg. Therefore, computer system 130 may prompt the clinician to reset the new visual field as the baseline, and may recommend follow up in six months with for a repeat visual field test. Moreover, the clinician may define or select alert criteria of providing an alert when the mean IOP is greater than 14 mm Hg, the glaucoma severity is low, and on an alert timeframe of when the patient chart is opened again. Furthermore, the clinician may specify a clinical question alert: Is the visual field loss progressing?

Additionally, after the follow-up, the visual field may continue to progress. Computer system 130 may note that there were no new interventions during the progression period and that the mean IOP has been 12 mm Hg. In response, the clinician may decide to perform surgery on the patient. Because the mean IOP is 12 mm Hg, minimally invasive procedures likely will not result in low enough IOP. Therefore, the clinician may proceed with a non-valved tube implant. Moreover, the clinician may instruct the patient to continue home monitoring, and may define or select post-operative alert criteria of providing an alert when the tube opens. For example, the alert criteria may include providing an alert when: a peak IOP greater than 22 mm Hg, glaucoma severity is high, and on an alert timeframe of the next day; a lowest IOP less than 7 mm Hg, glaucoma severity is high, and on an alert timeframe of the next day; and a drop in the mean IOP of greater than 20%, the glaucoma severity is high, and on an alert timeframe of the next day. In response, computer system 130 may provide an alert to the clinician when the IOP drops and the tube opens. This may allow the clinician to re-examine the patient to confirm that they are stable and that no intervention is required.

In some embodiments, one or more components in the system may provide additional or different services or capabilities. Notably, computer system 130 may combine relevant medical data acquired from patients (such as glaucoma-specific data that is acquired using a tonometer device) with information specifying a therapeutic intervention to provide clinical insights. For example, home eye pressure data may be combined with the timing of a therapy (such as when an eyedrop was prescribed or a surgery was performed) to determine efficacy of the therapy. Thus, in some embodiments, the monitoring techniques may incorporate therapeutics into the analysis to provide feedback as to how well a therapy impacted eye pressure. This may allow the monitoring techniques to be used to facilitate more effective clinical studies or trials of the therapy. Notably, the monitoring techniques may analyze the data in a therapeutic timeline and may provide summary or analysis information, such as an output to an electronic device, a printout or information stored in a computer-readable memory.

In some embodiments, the monitoring technique may use Natural Language Processing (NLP) to analyze electronic medical records (EMRs). For example, the NLP may be implemented using a pretrained neural network. This software may be used to analyze health record data (such as the EMRs), including physicians' notes, to structure and pull out when and what therapies were started (and, thus, their clinical impact). Notably, when a physician (and, more generally, a healthcare provider) writes in their clinical note ‘Start eyedrop X,’ the system may recognize this and place it on the therapeutic timeline. Then, eye pressure measurements prior to the start date to after the start date may be compared to determine therapeutic effect. A similar approach may be used when a type of surgery for glaucoma is performed or when changing one medication to another medication. Thus, the system may provide workflow tools and may generate clinical insights.

Moreover, because the system may not be 100% accurate, a physician may be dynamically kept in the loop to confirm some of the results or insights in the therapeutic timelines. For example, if the physician wrote something in an EMR that was difficult for the system to accurately interpret, it may confirm its interpretation by asking ‘Did the patient start eyedrop X on this date?’ with a link back to the source of the information. If the physician confirms the prompt, the system may generate the appropriate insights.

While the preceding examples illustrated embodiments where computer system 130 interacts with the clinician (i.e., a semi-automated approach), in other embodiments the monitoring techniques may perform some or all of the operations performed by the clinician (e.g., a more-automated or a fully automated approach, such as computer system 130 automatically resetting the baseline OCT or visual field tests after starting a new treatment).

We now describe embodiments of an electronic device, which may perform at least some of the operations in the monitoring techniques. FIG. 11 presents a block diagram illustrating an example of an electronic device 1100, e.g., monitoring device 110, electronic device 112, access points 116, radio node 118, switch 128, a computer or server in computer system 130, and/or computer 132, in accordance with some embodiments. For example, electronic device 1100 may include: processing subsystem 1110, memory subsystem 1112, and networking subsystem 1114. Processing subsystem 1110 includes one or more devices configured to perform computational operations. For example, processing subsystem 1110 can include one or more microprocessors, ASICs, microcontrollers, programmable-logic devices, GPUs and/or one or more DSPs. Note that a given component in processing subsystem 1110 are sometimes referred to as a ‘computation device’.

Memory subsystem 1112 includes one or more devices for storing data and/or instructions for processing subsystem 1110 and networking subsystem 1114. For example, memory subsystem 1112 can include dynamic random access memory (DRAM), static random access memory (SRAM), and/or other types of memory. In some embodiments, instructions for processing subsystem 1110 in memory subsystem 1112 include: program instructions or sets of instructions (such as program instructions 1122 or operating system 1124), which may be executed by processing subsystem 1110. Note that the one or more computer programs or program instructions may constitute a computer-program mechanism. Moreover, instructions in the various program instructions in memory subsystem 1112 may be implemented in: a high-level procedural language, an object-oriented programming language, and/or in an assembly or machine language. Furthermore, the programming language may be compiled or interpreted, e.g., configurable or configured (which may be used interchangeably in this discussion), to be executed by processing subsystem 1110.

In addition, memory subsystem 1112 can include mechanisms for controlling access to the memory. In some embodiments, memory subsystem 1112 includes a memory hierarchy that comprises one or more caches coupled to a memory in electronic device 1100. In some of these embodiments, one or more of the caches is located in processing subsystem 1110.

In some embodiments, memory subsystem 1112 is coupled to one or more high-capacity mass-storage devices (not shown). For example, memory subsystem 1112 can be coupled to a magnetic or optical drive, a solid-state drive, or another type of mass-storage device. In these embodiments, memory subsystem 1112 can be used by electronic device 1100 as fast-access storage for often-used data, while the mass-storage device is used to store less frequently used data.

Networking subsystem 1114 includes one or more devices configured to couple to and communicate on a wired and/or wireless network (i.e., to perform network operations), including: control logic 1116, an interface circuit 1118 and one or more antennas 1120 (or antenna elements). (While FIG. 11 includes one or more antennas 1120, in some embodiments electronic device 1100 includes one or more nodes, such as antenna nodes 1108, e.g., a metal pad or a connector, which can be coupled to the one or more antennas 1120, or nodes 1106, which can be coupled to a wired or optical connection or link. Thus, electronic device 1100 may or may not include the one or more antennas 1120. Note that the one or more nodes 1106 and/or antenna nodes 1108 may constitute input(s) to and/or output(s) from electronic device 1100.) For example, networking subsystem 1114 can include a Bluetooth™ networking system, a cellular networking system (e.g., a 3G/4G/5G network such as UMTS, LTE, etc.), a USB networking system, a networking system based on the standards described in IEEE 802.11 (e.g., a Wi-Fi® networking system), an Ethernet networking system, and/or another networking system.

Networking subsystem 1114 includes processors, controllers, radios/antennas, sockets/plugs, and/or other devices used for coupling to, communicating on, and handling data and events for each supported networking system. Note that mechanisms used for coupling to, communicating on, and handling data and events on the network for each network system are sometimes collectively referred to as a ‘network interface’ for the network system. Moreover, in some embodiments a ‘network’ or a ‘connection’ between electronic devices does not yet exist. Therefore, electronic device 1100 may use the mechanisms in networking subsystem 1114 for performing simple wireless communication between electronic devices, e.g., transmitting advertising or beacon frames and/or scanning for advertising frames transmitted by other electronic devices.

Within electronic device 1100, processing subsystem 1110, memory subsystem 1112, and networking subsystem 1114 are coupled together using bus 1128. Bus 1128 may include an electrical, optical, and/or electro-optical connection that the subsystems can use to communicate commands and data among one another. Although only one bus 1128 is shown for clarity, different embodiments can include a different number or configuration of electrical, optical, and/or electro-optical connections among the subsystems.

In some embodiments, electronic device 1100 includes a display subsystem 1126 for displaying information on a display, which may include a display driver and the display, such as a liquid-crystal display, a multi-touch touchscreen, etc. Moreover, electronic device 1100 may include a user-interface subsystem 1130, such as: a mouse, a keyboard, a trackpad, a stylus, a voice-recognition interface, and/or another human-machine interface.

Electronic device 1100 can be (or can be included in) any electronic device with at least one network interface. For example, electronic device 1100 can be (or can be included in): a desktop computer, a laptop computer, a subnotebook/netbook, a server, a supercomputer, a tablet computer, a smartphone, a smartwatch, a smart speaker, a cellular telephone, a consumer-electronic device, a portable computing device, communication equipment, a monitoring device and/or another electronic device.

Although specific components are used to describe electronic device 1100, in alternative embodiments, different components and/or subsystems may be present in electronic device 1100. For example, electronic device 1100 may include one or more additional processing subsystems, memory subsystems, networking subsystems, and/or display subsystems. Additionally, one or more of the subsystems may not be present in electronic device 1100. Moreover, in some embodiments, electronic device 1100 may include one or more additional subsystems that are not shown in FIG. 11. Also, although separate subsystems are shown in FIG. 11, in some embodiments some or all of a given subsystem or component can be integrated into one or more of the other subsystems or component(s) in electronic device 1100. For example, in some embodiments program instructions 1122 are included in operating system 1124 and/or control logic 1116 is included in interface circuit 1118.

Moreover, the circuits and components in electronic device 1100 may be implemented using any combination of analog and/or digital circuitry, including: bipolar, PMOS and/or NMOS gates or transistors. Furthermore, signals in these embodiments may include digital signals that have approximately discrete values and/or analog signals that have continuous values. Additionally, components and circuits may be single-ended or differential, and power supplies may be unipolar or bipolar.

An integrated circuit may implement some or all of the functionality of networking subsystem 1114 and/or electronic device 1100. The integrated circuit may include hardware and/or software mechanisms that are used for transmitting signals from electronic device 1100 and receiving signals at electronic device 1100 from other electronic devices. Aside from the mechanisms herein described, radios are generally known in the art and hence are not described in detail. In general, networking subsystem 1114 and/or the integrated circuit may include one or more radios.

In some embodiments, an output of a process for designing the integrated circuit, or a portion of the integrated circuit, which includes one or more of the circuits described herein may be a computer-readable medium such as, for example, a magnetic tape or an optical or magnetic disk or solid state disk. The computer-readable medium may be encoded with data structures or other information describing circuitry that may be physically instantiated as the integrated circuit or the portion of the integrated circuit. Although various formats may be used for such encoding, these data structures are commonly written in: Caltech Intermediate Format (CIF), Calma GDS II Stream Format (GDSII), Electronic Design Interchange Format (EDIF), OpenAccess (OA), or Open Artwork System Interchange Standard (OASIS). Those of skill in the art of integrated circuit design can develop such data structures from schematics of the type detailed above and the corresponding descriptions and encode the data structures on the computer-readable medium. Those of skill in the art of integrated circuit fabrication can use such encoded data to fabricate integrated circuits that include one or more of the circuits described herein.

While some of the operations in the preceding embodiments were implemented in hardware or software, in general the operations in the preceding embodiments can be implemented in a wide variety of configurations and architectures. Therefore, some or all of the operations in the preceding embodiments may be performed in hardware, in software or both. For example, at least some of the operations in the monitoring techniques may be implemented using program instructions 1122, operating system 1124 (such as a driver for interface circuit 1118) or in firmware in interface circuit 1118. Thus, the monitoring techniques may be implemented at runtime of program instructions 1122. Alternatively or additionally, at least some of the operations in the monitoring techniques may be implemented in a physical layer, such as hardware in interface circuit 1118.

In the preceding description, we refer to ‘some embodiments’. Note that ‘some embodiments’ describes a subset of all of the possible embodiments, but does not always specify the same subset of embodiments. Moreover, note that the numerical values provided are intended as illustrations of the monitoring techniques. In other embodiments, the numerical values can be modified or changed.

The foregoing description is intended to enable any person skilled in the art to make and use the disclosure, and is provided in the context of a particular application and its requirements. Moreover, the foregoing descriptions of embodiments of the present disclosure have been presented for purposes of illustration and description only. They are not intended to be exhaustive or to limit the present disclosure to the forms disclosed. Accordingly, many modifications and variations will be apparent to practitioners skilled in the art, and the general principles defined herein may be applied to other embodiments and applications without departing from the spirit and scope of the present disclosure. Additionally, the discussion of the preceding embodiments is not intended to limit the present disclosure. Thus, the present disclosure is not intended to be limited to the embodiments shown, but is to be accorded the widest scope consistent with the principles and features disclosed herein.

What is claimed is:

1. A computer system, comprising:
an interface circuit;
a computation device coupled to the interface circuit; and
memory, coupled to the computation device, storing program instructions, wherein, when executed by the computation device, the program instructions cause the computer system to perform one or more operations comprising:
obtaining a set of data associated with an individual, wherein the set of data comprises or corresponds to a history of glaucoma diagnostic or glaucoma treatment data for the individual during a time interval;
determining whether the set of data is medically meaningful for the individual based at least in part on one or more of: a number of data instances in the set of data, a number of types of data in the set of data, or sampling times of the data instances;
when the set of data is determined to not be medically meaningful for the individual, providing, addressed to an electronic device, a request for additional data, wherein the additional data comprises one or more of: an additional data instance, an additional type of data that is different from the types of data, or additional glaucoma diagnostic or glaucoma treatment data at one or more different sampling times than the sampling times of the data instances; and
when the set data is determined to be medically meaningful for the individual:
analyzing the set of data to compute analysis results; and
selectively providing, addressed to the electronic device, a second electronic device or both, feedback associated with glaucoma of the individual or glaucoma treatment of the individual, wherein the feedback is selectively provided based at least in part on analysis results.

2. The computer system of claim 1, wherein the obtaining comprises performing measurements of the glaucoma diagnostic or the glaucoma treatment data.

3. The computer system of claim 1, wherein the obtaining comprises accessing the glaucoma diagnostic or the glaucoma treatment data stored in the memory.

4. The computer system of claim 1, wherein the obtaining comprises receiving, associated with the electronic device, the glaucoma diagnostic or the glaucoma treatment data.

5. The computer system of claim 1, wherein the electronic device is associated with the individual and the second electronic device is associated with a clinician.

6. The computer system of claim 1, wherein the determining, the analyzing or both are performed using a pretrained model.

7. The computer system of claim 6, wherein the pretrained model comprises a supervised-learning model or a neural network.

8. The computer system of claim 1, wherein the feedback comprises a treatment recommendation.

9. The computer system of claim 1, wherein the determining, the analysis or both is based at least in part on one or more of: a glaucoma stage or risk of the individual; a prescribed medication used by the individual; one or more surgical, medical, or laser treatments or interventions performed on an individual, a clinician-defined alert criterion, a clinical insight or question, or demographic information of the individual.

10. The computer system of claim 1, wherein the types of data comprise:
intraocular pressure, a visual field test, Optical Coherence Tomography (OCT), pachymetry, corneal hysteresis, a prescribed medication, a surgical or laser intervention performed by a clinician, a patient-supplied input, medication compliance of the individual, or times when a prescribed medication is used by the individual.

11. A non-transitory computer-readable storage medium for use in conjunction with a computer system, the computer-readable storage medium configured to store program instructions that, when executed by the computer system, causes the computer system to perform one or more operations comprising:
obtaining a set of data associated with an individual, wherein the set of data comprises or corresponds to a history of glaucoma diagnostic or glaucoma treatment data for the individual during a time interval;
determining whether the set of data is medically meaningful for the individual based at least in part on one or more of: a number of data instances in the set of data, a number of types of data in the set of data, or sampling times of the data instances;
when the set of data is determined to not be medically meaningful for the individual, providing, addressed to an electronic device, a request for additional data, wherein the additional data comprises one or more of: an additional data instance, an additional type of data that is different from the types of data, or additional glaucoma diagnostic or glaucoma treatment data at one or more different sampling times than the sampling times of the data instances; and
when the set data is determined to be medically meaningful for the individual:
analyzing the set of data to compute analysis results; and
selectively providing, addressed to the electronic device, a second electronic device or both, feedback associated with glaucoma of the individual or glaucoma treatment of the individual, wherein the feedback is selectively provided based at least in part on analysis results.

12. The non-transitory computer-readable storage medium of claim 11, wherein the obtaining comprises: performing measurements of the glaucoma diagnostic or the glaucoma treatment data; accessing the glaucoma diagnostic or the glaucoma treatment data stored in the memory; or receiving, associated with the electronic device, the glaucoma diagnostic or the glaucoma treatment data.

13. The non-transitory computer-readable storage medium of claim 11, wherein the electronic device is associated with the individual and the second electronic device is associated with a clinician.

14. The non-transitory computer-readable storage medium of claim 11, wherein the determining, the analyzing or both are performed using a pretrained model.

15. The non-transitory computer-readable storage medium of claim 11, wherein the determining, the analysis or both is based at least in part on one or more of: a glaucoma stage or risk of the individual; a prescribed medication used by the individual; one or more surgical, medical, or laser treatments or interventions performed on an individual, a clinician-defined alert criterion, a clinical insight or question, or demographic information of the individual.

16. A method for selectively providing feedback, comprising by a computer system:
obtaining a set of data associated with an individual, wherein the set of data comprises or corresponds to a history of glaucoma diagnostic or glaucoma treatment data for the individual during a time interval;
determining whether the set of data is medically meaningful for the individual based at least in part on one or more of: a number of data instances in the set of data, a number of types of data in the set of data, or sampling times of the data instances;
when the set of data is determined to not be medically meaningful for the individual, providing, addressed to an electronic device, a request for additional data, wherein the additional data comprises one or more of: an additional data instance, an additional type of data that is different from the types of data, or additional glaucoma diagnostic or glaucoma treatment data at one or more different sampling times than the sampling times of the data instances; and
when the set data is determined to be medically meaningful for the individual:
analyzing the set of data to compute analysis results; and
selectively providing, addressed to the electronic device, a second electronic device or both, the feedback associated with glaucoma of the individual or glaucoma treatment of the individual, wherein the feedback is selectively provided based at least in part on analysis results.

17. The method of claim 16, wherein the obtaining comprises: performing measurements of the glaucoma diagnostic or the glaucoma treatment data; accessing the glaucoma diagnostic or the glaucoma treatment data stored in the memory; or receiving, associated with the electronic device, the glaucoma diagnostic or the glaucoma treatment data.

18. The method of claim 16, wherein the electronic device is associated with the individual and the second electronic device is associated with a clinician.

19. The method of claim 16, wherein the determining, the analyzing or both are performed using a pretrained model.

20. The method of claim 16, wherein the determining, the analysis or both is based at least in part on one or more of: a glaucoma stage or risk of the individual; a prescribed medication used by the individual; one or more surgical, medical, or laser treatments or interventions performed on an individual, a clinician-defined alert criterion, a clinical insight or question, or demographic information of the individual.
